(19) United States
(12) Patent Application Publication
(10) Pub. No.: US 2026/0356909 A1
(43) Pub. Date: Nov. 12, 2026

(54) CD47 MUTANT AND ITS APPLICATIONS

(71) Applicant: Xeus Biosciences Shanghai Limited, Shanghai (CN)

(72) Inventors: Xiaodan Wang, Shanghai (CN); Yongguang Yang, Shanghai (CN); Lu Xu, Shanghai (CN)

(21) Appl. No.: 19/666,096

(22) Filed: May 1, 2026

Related U.S. Application Data
(63) Continuation of application No. PCT/CN2024/129458, filed on Nov. 1, 2024.

(30) Foreign Application Priority Data
Nov. 3, 2023 (CN) 202311457148.3

(51) Int. Cl. C07K 14/705; A01K 67/0278; A61K 35/28; A61K 35/39; A61K 35/545

(52) U.S. Cl. CPC C07K 14/70503; A01K 67/0278; A61K 35/28; A61K 35/39; A61K 35/545; A01K 2207/15; A01K 2217/052; A01K 2227/106; A01K 2227/108; A01K 2267/025

(57) ABSTRACT

Provided is a CD47 mutant and its applications, wherein the CD47 mutant comprises a partial amino acid sequence of CD47 and an amino acid sequence of a glycosylphosphatidylinositol (GPI) attachment signal. The CD47 mutant is obtained by removing sequences (transmembrane and intracellular regions) potentially involved in transduction of signals with negative effects such as inhibiting cell survival, suppressing blood flow and angiogenesis, as well as promoting tissue injury. Simultaneously, beneficial functional segment (extracellular IgV domain) of the CD47 protein sequence, which is involved in inhibiting phagocytosis and cytotoxic activity of myeloid cells, is fused with GPI membrane anchor attachment signal sequence from human decay-accelerating factor (DAF) protein. The obtained CD47 mutant can protect cell and organ grafts from transplant rejection mediated by myeloid cells and other SIRPα-positive immune cells, with efficacy comparable to wild-type CD47, while eliminating negative effects of wild-type CD47.

### CROSS-REFERENCE TO RELATED APPLICATIONS

This application is a continuation of International Patent Application No. PCT/CN2024/129458 filed Nov. 1, 2024, which claims priority to and the benefit of Chinese Patent Application No. 202311457148.3 filed Mar. 11, 2023. The contents of International Patent Application No. PCT/CN2024/129458 are incorporated by reference herein in their entirety.

### REFERENCE TO SEQUENCE LISTING XML

This application contains a Sequence Listing which has been submitted electronically in XML format. The Sequence Listing XML is incorporated herein by reference. Said XML file, created on Apr. 30, 2026, is named XEUS-001USWOC1_SL.xml and is 37,954 bytes in size.

### BACKGROUND OF THE APPLICATION

### 1. Technical Field

The present disclosure generally relates to molecular immunology, and more specifically to a CD47 mutant and its applications. In particular, the present disclosure relates to a CD47 mutant that has eliminated the negative effects of wild-type CD47, such as inhibitory effects on cell survival and angiogenesis, while retaining its beneficial functions of inhibiting transplant rejection mediated by recipient myeloid cells and other SIRPα-positive immune cells. The present disclosure also encompasses various clinical translational uses of the said mutant in the field of transplantation.

### 2. Description of Related Art

CD47, also known as integrin-associated protein, is a glycosylated protein with five transmembrane domains that is widely expressed on the surface of various human cells. It is a member of the immunoglobulin superfamily. CD47 consists of an extracellular Immunoglobulin variable (IgV) domain that interacts with corresponding ligands, a highly hydrophobic region with five transmembrane segments, and a high hydrophilic carboxyl-terminal cytoplasmic tail.

Cell and organ transplantation are the most effective means to sustain the lives of many critically ill patients, such as those with end-stage organ failure. Organ transplantation involves implanting a healthy organ from a donor into a recipient to replace a diseased organ and quickly restore function. There has long been a severe shortage of organ donors. The cultivation and application of transgenic donor animals for xenotransplantation have thus emerged as one of the most promising strategies to address this issue in the future. As of now, two FDA-approved (compassionate use) clinical cases of pig-to-human heart xenotransplantation and two clinical cases of pig-to-human kidney xenotransplantation have been performed in the United States. However, compared with allotransplantation, xenotransplantation faces more rapid, intense, and persistent rejection reactions due to factors such as species differences in antigens, incompatibility between immune suppressive receptors and ligands across species, and coagulation disorders between species. Additionally, in both allotransplantation and xenotransplantation, transplant injury induced by unavoidable factors such as organ ischemia-reperfusion during surgery seriously threatens the survival and function of the graft. Therefore, researching and modifying species-specific molecules related to immune rejection of the xenotransplant and molecules promoting transplant injury that affect the function and survival of allografts and xenografts, and applying the results to optimize the generation of transgenic donor animals for xenotransplantation or the transgenic modification of allogeneic cell grafts, has broad application prospects and high clinical translation value in the field of transplantation.

CD47 exhibits dual and opposing functions in the field of transplantation. Beneficially, CD47 serves as a “self-marker” recognized by the immune system, thereby protecting cells from immune clearance. Specifically, its extracellular IgV domain, with the assistance of its transmembrane region, can species-specifically bind and activate the immunosuppressive receptor SIRPα (Signal Regulatory Protein a) on myeloid cells and other SIRPα-positive immune cells, inhibiting their immune functions. This includes inhibiting the phagocytic and cytotoxic activities of macrophages (Oldenborg, P A et al. 2000. Science 288, 2051-2054; Navarro-Alvarez, N & Yang, Y G. 2014. Cell Transplant 23, 345-354; Rebres, R A et al. 2001. J Biol Chem 276, 34607-34616) and suppressing antigen-uptake capacity of dendritic cells and its activation, thereby inhibiting the recruitment and activation of natural killer and T cells (Wang, S et al. 2022. J Hepatol 77, 467-478; Autio, A et al. 2022. PLoS One 17, e0266566; Li, Y et al. 2020. Nat Commun 11, 581). Ultimately, this suppresses transplant rejection mediated by myeloid cells and other SIRPα-positive immune cells. The “self-marker” function of CD47 makes it one of the key species-specific molecules related to immune rejection of the xenotransplant (Navarro-Alvarez, N & Yang, Y G. 2011. Cell Mol Immunol 8, 285-288; Maeda, A et al. 2020. Transplantation 104, 675-681). Conversely, CD47 can exert negative effects when activated by Thrombospondin-1 (TSP-1), soluble SIRPα, CD47 agonistic antibodies, and other undefined ligands. Such activation can transduce multiple signals of inhibitory effects on cell survival into cells, inhibiting angiogenesis and blood flow, and ultimately exacerbate transplant rejection and injury of cell and organ grafts (Roberts, D D et al. 2012. Matrix Biol 31, 162-169; Rogers, N M et al. 2014. Matrix Biol 37, 92-101; Soto-Pantoja, D R et al. 2015. Crit Rev Biochem Mol Biol 50, 212-230). Several research teams have reported that the blockade, down-regulation, or absence of CD47 significantly alleviates graft injury, particularly those caused by ischemia-reperfusion injury, thereby improving graft survival rates and function (Chen, M et al. 2019. Xenotransplantation 26, e12459; Garcia-Aroz, S et al. 2022. Transplantation 106, 37-47; Isenberg, J S & Roberts, D D. 2019. Pediatr Nephrol 34, 2479-2494). Thus, existing studies indicate that CD47 possesses both beneficial species-restricted anti-rejection capability and adverse non-species-restricted capacity to exacerbate transplant injury. As a result, the modification of wild-type CD47 through mutation and the functional studies of its various domains have become one of the focal areas of research in the field of transplantation.

Current reports suggest the primary known pathways through which CD47 inhibits graft survival are as follows: 1) Activated CD47 can trigger Gᵢ protein signaling, which reduces intracellular cAMP levels and inhibits calcium flux, ultimately suppressing cell survival (Frazier; W A et al. 1999. J Biol Chem 274, 8554-8560); 2) Activated CD47 can inhibit graft survival by suppressing the pro-survival NO/cGMP signaling (Isenberg, J S et al. 2008. Cell Mol Life Sci 65, 728-742); 3) Activated CD47 can inhibit angiogenesis and blood flow by antagonizing vascular endothelial growth factor (VEGF) signaling (Kaur; S et al. 2010. J Biol Chem 285, 38923-38932); 4) Activated CD47 can induce cell death by initiating the release of cytochrome C from mitochondria via BNIP3 (Zhang, J & Ney, PA. 2009. Cell Death Differ 16, 939-946); and 5) Activated CD47 can reduce the limitation of eNOS activity by Cav-1, causing pathological eNOS hyperactivity, generating ROS, and leading to tissue injury (Bauer; P M et al. 2012. Cardiovasc Res 93, 682-693). Researchers have supposed, based on experimental data regarding the interactions between CD47 and key proteins in these pathways, as well as the structure and localization of related proteins, that the aforementioned negative effects of leading to cell dysfunction, inhibiting cell survival, blood flow, angiogenesis, and promoting tissue injury are likely mediated by the participation of CD47 transmembrane and intracellular regions (Rogers, N M et al. 2014. Matrix Biol 37, 92-101; Soto-Pantoja, D R et al. 2013. Expert Opin Ther Targets 17, 89-103; Frazier, W A et al. 1999. J Biol Chem 274, 8554-8560; Lamy, L et al. 2003. J Biol Chem 278, 23915-23921; Bauer, P M et al. 2012. Cardiovasc Res 93, 682-693).

Pigs are widely recognized as the most suitable donor animals for xenotransplantation to date (Cooper; D K et al. 2002. Annu Rev Med 53, 133-147). However, pig CD47 cannot effectively activate SIRPα on human or non-human primate myeloid cells and other SIRPα-positive immune cells, thus failing to protect porcine cells and organ grafts from immune rejection of the xenotransplant mediated by myeloid cells and other SIRPα-positive immune cells (Navarro-Alvarez, N & Yang, Y G. 2014. Cell Transplant 23, 345-354; Wang, H et al. 2007. Blood 109, 836-842; Cooper, D K C et al. 2019. Xenotransplantation 26, e12516). Researchers have already combined human CD47 with several other species-specific molecules related to xenotransplantation and transgenically introduced them into the porcine genome. They found that the expression of human CD47 could relatively extend the survival duration of transgenic porcine cells and organ grafts in non-human primates. Based on these findings, it is broadly recognized that overexpression of human CD47 on porcine cell membrane can protect porcine grafts from immune rejection mediated by human myeloid cells and other SIRPα-positive immune cells (Takeuchi, K et al. 2021. Xenotransplantation 28, e12708; Tena, A A et al. 2017. Transplantation 101, 316-321; Watanabe, H et al. 2020. Xenotransplantation 27, e12552). However, due to its negative effects as mentioned earlier, CD47 inhibits angiogenesis and mediates and exacerbates graft injury in the inflammatory microenvironment caused by transplant surgery. Therefore, direct transgenic overexpression of human wild-type CD47 in donor animals for xenotransplantation according to the current general protocol cannot maximize the survival time of the grafts and cannot effectively protect the physiological functions of the grafts.

Additionally, CD47 plays a significant role not only in xenotransplantation but also in preventing allogeneic transplant rejection, as studies have shown that overexpressing CD47 can be used for this purpose (Deuse, T et al. 2019. Nat Biotechnol 37, 252-258). Beyond xenotransplantation therapies, the utilization of various cell products, including autologous or allogeneic stem cells, provides an unlimited source of cells for tissue and organ repair based on specific cells. Allogeneic cell products have several advantages over autologous ones, such as faster preparation, stable quality, and lower costs. However, the strong immune rejection response of recipients against histoincompatible cells hinders the successful implementation of this approach (van Berlo, J H & Molkentin, J D. 2014. Nat Med 20, 1386-1393). Recent research has found that overexpressing CD47 can render human cell grafts hypoimmunogenic in the context of major histocompatibility complex genes inactivation, thus successfully avoiding immune rejection against fully MHC-mismatched allogeneic grafts in recipients without the need for immunosuppressive measures, allowing stem cell grafts or their derived cell grafts to survive long-term (Deuse, T et al. 2019. Nat Biotechnol 37, 252-258). It is noteworthy that overexpressing wild-type CD47 also has inhibitory effects on the survival and function of cell grafts in the inflammatory microenvironment, which caused by the transplant procedures and the pathological state of the organ to be repaired. Recent reports indicate that the expression of wild-type CD47 can inhibit insulin secretion, impair pancreatic β-cell function, and affect the transplantation outcome of islets (Ghimire, K et al. 2023. Sci Transl Med 15, eadd2387). Due to these drawbacks, overexpressing wild-type CD47 is not the optimal strategy for producing therapeutic transgenic cell products.

As previously mentioned, the negative effects of wild-type CD47 in transplantation are generally recognized in existing technologies to be mediated by the participation of its transmembrane and intracellular domains. Concurrently, research results in the current field broadly agree that the transmembrane domain of CD47 and a key disulfide bond between the transmembrane domain and the extracellular IgV domain are essential for binding and activating the receptor SIRPα to exert CD47's beneficial anti-rejection effects (Rebres, R A et al. 2001. J Biol Chem 276, 34607-34616). Although a modified soluble IgV domain mutant of CD47, which lacks the transmembrane and intracellular regions, can bind to SIRPα with high affinity, it cannot activate SIRPα and is instead used to block SIRPα signaling, thereby impeding SIRPα's inhibition of myeloid cell phagocytosis (Ho, C C et al. 2015. J Biol Chem 290, 12650-12663). Furthermore, fusion membrane proteins composed of the CD47 IgV domain and a type I transmembrane helix from other functionally irrelevant proteins (serving a membrane localization function similar to a GPI membrane anchor) exhibit poor binding to SIRPα (approximately half the binding affinity of the wild type) and are presumed incapable of activating SIRPα (Rebres, R A et al. 2001. J Biol Chem 276, 34607-34616). Subsequent research has failed to identify the exact motifs and sites in CD47 responsible for mediating inhibitory effects on cell survival. Thus, although the transmembrane and intracellular regions of CD47 are likely key structural domains involved in mediating its negative effects, these regions are also critical for its beneficial functions. Therefore, simple deletions, replacements, or precise mutations in these regions cannot be conducted. Consequently, CD47 modification work in this field has stalled.

Fenalti, Gustavo, et al. “Structure of the human marker of self 5-transmembrane receptor CD47.” Nature communications 12.1 (2021): 5218. cites the human CD47 protein, which has the following amino sequence:

(SEQ ID No. 27)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFVASNQKTIQPPRKAVEEPLNAFKE
SKGMMNDE
(see “Source data”).

Caras, Ingrid W., et al. “Signal for attachment of a phospholipid membrane anchor in decay accelerating factor.” Science 238.4831 (1987): 1280-1283. discloses the 37 amino acids from the carboxyl terminus of the decay-accelerating factor (DAF), which is: PNKGSGTTSGTTRLLSGHTCFTLTGLLGTLVTMGLLT (SEQ ID No. 7) (see FIG. 1b of the cited article Caras, Ingrid W., et al.).

Lindberg, Frederik P., et al. “Integrin-associated protein immunoglobulin domain is necessary for efficient vitronectin bead binding.” The Journal of cell biology 134.5 (1996): 1313-1322. discloses a CD47 mutant which is obtained by connecting the IgV structure of CD47 with the GPI structure of DAF, see the IAP-GPI structure shown in FIG. 1 of the cited article Lindberg, Frederik P., et al.

It should be noted that there may be discrepancies between the existing art understood by the applicant and that known to patent examiners. Furthermore, due to space limitations, not all details and content from the numerous literature and patent documents referenced by the applicant during the development of the present disclosure have been exhaustively recited herein. However, this does not imply that the present disclosure lacks these features of the existing art. On the contrary, the present disclosure incorporates all relevant features of the existing art. The applicant reserves the right to supplement the background section with additional relevant features of the existing art as support, in accordance with applicable regulations.

### SUMMARY OF THE APPLICATION

The present disclosure addresses the aforementioned drawbacks of wild-type CD47 and existing CD47 soluble IgV domain mutants. In an effort to eliminate the negative effects of CD47 while retaining its beneficial functions, thereby advancing its transgenic application in the field of transplantation, the applicant has engineered the CD47 protein. Unexpectedly, it was discovered that GPI+CD47 IgV can effectively activate the immunosuppressive function of SIRPα. This discovery led to the successful development of a CD47 mutant that protects cell and organ grafts from myeloid cell-mediated transplant rejection with efficacy comparable to wild-type CD47, while avoiding the inhibitory effects on angiogenesis and the mediation and exacerbation of cell and organ graft injuries associated with wild-type CD47. The objective of the present disclosure is to provide a CD47 mutant possessing aforementioned advantages and its applications in enhancing graft survival rate and function, as well as reducing graft immunogenicity.

The present disclosure achieves the aforementioned objectives through the following approach: The five transmembrane domain sequences and intracellular region (including intracellular loop and carboxyl-terminal) sequences of human CD47, which are potentially involved in mediating interactions between CD47 and the key proteins in the pathways aggravating graft injury, as well as mediating signal transduction across membranes leading to inhibitory effects on cell survival and blood flow and angiogenesis, are removed. Only the signal peptide and extracellular immunoglobulin variable-like (IgV) domain of CD47 (Fenalti, G et al. 2021. Nat Commun 12, 5218) (specifically, residues 1-137, SEQ ID NO.5) are retained. This retained sequence is then fused with the glycosylphosphatidylinositol (GPI) attachment signal sequence (also known as GPI anchoring signal sequence, SEQ ID NO.7) composed of 37 amino acids from the carboxyl terminus of the decay-accelerating factor (DAF), resulting in a fusion protein (i.e., a CD47 mutant). When the GPI attachment signal sequence is fused to the carboxyl terminus of the protein of the retained sequence of CD47, it can direct the covalent addition of a glycosylphosphatidylinositol membrane anchor (also known as a glycophospholipid membrane anchor) to the carboxyl terminus of the fusion protein, while the membrane anchor is sufficient to target the fusion protein for transport to the cell membrane surface (Caras, I W et al. 1987. Science 238, 1280-1283).

The cDNAs of the aforementioned CD47 mutant and its wild-type counterpart (isoform 2, the most widely expressed CD47 isoform) were separately cloned into the pRRLSIN.cPPT.MSCV.WPRE lentiviral vector (human wild-type CD47 coding sequence is SEQ ID NO.13, and human mutant CD47 coding sequence is SEQ ID NO.8). Lentiviruses were produced and used to transduce human T-lymphoma cell line Jurkat, human endothelial cell line EA.hy926, and porcine epithelial cell line PK15, which are deficient in endogenous CD47. Cell lines stably expressing the CD47 mutant or wild-type control with the lentiviral vectors integrated into their genomes were obtained by flow sorting. To test whether the mutant CD47 ligation induce inhibitory effects on cell survival, Jurkat cells stably expressing the mutant or wild-type control were treated with or without 250 ng/ml CC2C6 antibody (an agonist anti-CD47 antibody used to induce CD47-specific apoptosis of cells) for 2 hours. AnnexinV binding and PI uptake levels were then detected by flow cytometry to assess the apoptosis induction of wild-type and mutant CD47 in vitro, resulting in the identification of a mutant that no longer mediates apoptosis like wild-type CD47. To test the ability of the mutant to inhibit macrophage phagocytosis, human peripheral blood mononuclear cells (PBMCs) were isolated from blood of healthy volunteers by Ficoll density gradient centrifugation and used to establish an in vitro assessment system for phagocytosis. PBMCs were cultured with 20 ng/ml human macrophage colony-stimulating factor (M-CSF) for 7-9 days to obtain functional human macrophages. CFSE-labeled CD47KO Jurkat cells and CD47KO Jurkat cells stably expressing the human mutant (hCD47-IgV) or wild-type control (hCD47-iso2) were then co-incubated with human macrophages for 4 hours. Macrophages were stained with anti-human CD172α/β (SIRPα/β)-APC antibody. The percentages of SIRPα⁺CFSE⁺ double-positive macrophages that had phagocytosed Jurkat cells were detected by flow cytometry to assess phagocytosis. Additionally, due to the limited source of human blood, macrophages derived from NCG mice, whose SIRPα can cross-react with human CD47, were also used to establish the phagocytosis assessment system for extensive confirmatory experiments. Bone marrow cells from NCG mice were incubated with 1640 complete medium containing 20 ng/ml mouse M-CSF for 7-9 days, and then activated with 1640 complete medium containing 20 ng/ml LPS for 8-12 hours to prepare mouse macrophages. The CFSE-labeled Jurkat target cells of each group were co-incubated with these macrophages for 4 hours. Macrophages were stained with anti-mouse F4/80-APC antibody, and the percentages of F4/80⁺CFSE⁺ macrophages were detected by flow cytometry and confocal microscopy to assess phagocytosis.

A CD47 mutant with phagocytosis inhibitory ability equivalent to that of wild-type CD47 was obtained and validated through the two assessment systems for phagocytosis described above. To explore the feasibility of applying such mutant to the genetic modification of donor animals (such as pigs) for improving xenotransplantation outcomes, the present disclosure compared the phagocytosis levels of porcine CD47KO PK15 cells and CD47KO PK15 cells stably expressing the human mutant (hCD47-IgV) by human macrophages using the aforementioned phagocytosis assessment system in vitro. Target cells were labeled with PKH67 and co-incubated with macrophages for 4 hours, subsequently, macrophages were stained with anti-human CD172α/β (SIRPα/β)-APC antibody, and the percentages of SIRPα⁺PKH67⁺ double-positive macrophages that had phagocytosed PK15 cells (PKH67⁺) were detected and compared by flow cytometry. This resulted in the identification of a mutant capable of protecting porcine cells against phagocytosis by human macrophages.

To test whether the phagocytosis inhibitory ability conferred by the structural features of the CD47 mutant of the present disclosure is a species-specific phenomenon, a mouse CD47 mutant (SEQ ID NO.2) structurally corresponding to the human mutant was constructed and its function was evaluated. Corresponding to functional domains of the human CD47 mutant, sequences of the transmembrane and intracellular regions of the mouse CD47 were removed and the retained sequence of signal peptide and IgV domain (specifically, residues 1-161aa, SEQ ID NO.6) was fused with the GPI attachment signal sequence (SEQ ID NO.7) to generate the mouse CD47 mutant, also referred to as the mouse mutant CD47. The cDNAs of the mouse wild-type and mutant CD47 were separately cloned into the pRRLSIN.cPPT.MSCV.WPRE lentiviral vector (mouse wild-type CD47 coding sequence is SEQ ID NO.14, and mouse mutant CD47 coding sequence is SEQ ID NO.9). Lentiviruses were produced and used to transduce mouse A20 cells deficient in endogenous CD47 and wild-type A20 cells. Cell lines stably expressing the mouse CD47 mutant (mCD47-IgV) and wild-type control (mCD47-iso2) with the lentiviral vectors integrated into their genomes were obtained by flow sorting.

Using macrophages derived from BALB/c mice bone marrow, which are syngeneic with A20 cells, an in vitro assessment system for phagocytosis was established as described above to test the protection ability against phagocytosis of CD47KO A20 cells provided by the transgenic expression of mCD47-IgV and mCD47-iso2. Studies have shown that the results obtained in the mouse cell system are consistent with those in the human cell system, with the mouse CD47 mutant having the same level of inhibitory ability of macrophage phagocytosis as wild-type CD47 in vitro.

The present disclosure further tested the leukemogenic potential of CD47KO A20 cells and CD47KO A20 cells stably expressing mCD47-IgV or mCD47-iso2 using a mouse tumor cell transplantation model in vivo. The results showed that the mouse CD47 mutant and its wild-type CD47 control could protect CD47KO A20 tumor cells from macrophage-mediated transplant rejection to the same significant extent in vivo. This indicates that CD47 from different species engineered with the structural features provided by the present disclosure can confer resistance against phagocytosis by macrophages of corresponding species to cells.

Due to the transgenic expression of the CD47 mutant and wild-type control in CD47KO A20 cells being considerably weaker than the endogenous CD47 expression in the parental wild-type A20 cells, this may make the negative effects of the transgenic CD47 undetectable. Therefore, the present disclosure also tested the leukemogenic potential of CD47KO A20 cells, wild-type A20 cells, and wild-type A20 cells further stably overexpressing mCD47-IgV (mCD47-IgV/WT) or mCD47-iso2 (mCD47-iso2/WT) on the basis of endogenous CD47 expression using a mouse tumor cell transplantation model in vivo. The results showed that overexpressing CD47-IgV in wild-type cells could further enhance the anti-phagocytic ability of wild-type cells, thereby increasing the in vivo survival rate of transplanted mCD47-IgV/WT transgenic A20 cells. However, overexpressing CD47-iso2 may have inhibited cell survival due to its transmission of death signals at such a high expression level, and thus no improvement in the in vivo survival rate of mCD47-iso2/WT transgenic A20 cells was observed. This in vivo detection confirms that transgenic expression of the CD47 mutant provides a higher survival rate for grafts compared with wild-type CD47.

On the other hand, since the CD47 mutant provided by the present disclosure also has potential applications in the preparation of hypoimmunogenic allogeneic cell therapy products, the present disclosure sought to evaluate whether the transgenic expression of such mutant in various cell products, represented by hematopoietic stem cells, could protect cell grafts from transplant rejection mediated by myeloid cells and other SIRPα-positive immune cells as effectively as wild-type in vivo. Additionally, the study aimed to determine whether the transgenic expression of such mutant may affect the function of cell grafts. To this end, the present disclosure examined and compared the ability of CD47KO GFP mouse hematopoietic stem/progenitor cells transduced with the mouse CD47 mutant or wild-type CD47 to engraft and differentiate in syngeneic wild-type recipients. As a result, a CD47 mutant was obtained that can inhibit transplant rejection in vivo without affecting the function and differentiation of cell grafts, as exemplified by hematopoietic stem cells.

Furthermore, as is known that activated wild-type CD47 has negative effects of inhibiting angiogenesis and blood flow, the present disclosure evaluated the effect of transgenic expression of the CD47 mutant provided by the present disclosure on angiogenesis. This was accomplished by examining and comparing the in vivo and in vitro angiogenic capabilities of CD47KO EA.hy926 endothelial cells stably expressing the human CD47 mutant and wild-type CD47 control. It has been reported that endothelial cells with endogenous wild-type CD47 expression undergo senescence and angiogenic impairment during continuous passaging, whereas endothelial cells knocked out for CD47 do not exhibit this senescence-related reduction in angiogenesis ability. The present disclosure also investigated whether the transgenic expression of the CD47 mutant would lead to the aforementioned phenomenon. Through these assessments, it was confirmed that the CD47 mutant obtained in the present disclosure doesn't inhibit angiogenesis in vivo and in vitro, and also mitigates the angiogenic impairment caused by senescence of endothelial cells during continuous passaging.

The first aspect of the present disclosure provides a CD47 mutant, which comprises a partial amino acid sequence of CD47 and an amino acid sequence of a glycosylphosphatidylinositol (GPI) attachment signal.

According to a preferred embodiment, the CD47 mutant is generated by removing amino acid sequence of transmembrane region and intracellular region of wild-type CD47, while adding the amino acid sequence of GPI membrane anchor attachment signal, so as to eliminate negative effects of wild-type CD47 promoting graft injury.

According to a preferred embodiment, the CD47 mutant is obtained by fusing the partial amino acid sequence of CD47 with the amino acid sequence of the GPI attachment signal.

According to a preferred embodiment, the partial amino acid sequence of CD47, starting at the amino terminus, comprises its signal peptide and extracellular IgV domain sequence.

According to a preferred embodiment, the amino acid sequence of the GPI attachment signal is fused to the carboxyl terminus of the CD47 protein, thereby enabling the mutant to possess a GPI membrane anchor.

According to a preferred embodiment, the GPI attachment signal consists of 37 amino acids from the carboxyl terminus of the decay-accelerating factor (DAF). Specifically, the GPI attachment signal is derived from the last 37 amino acids at the carboxyl terminus of the DAF.

According to a preferred embodiment, the GPI membrane anchor is a GPI anchor specifically enriched in lipid rafts.

According to a preferred embodiment, the GPI anchor specifically enriched in lipid rafts refers to an anchor that can direct the transport of the majority of the fusion protein to localize to the lipid rafts in the cell membrane.

According to a preferred embodiment, the CD47 mutant achieves proper intracellular trafficking and high expression on the outer leaflet of the cell membrane via the fused GPI membrane anchor.

According to a preferred embodiment, the CD47 mutant, when overexpressed in donor cells, inhibits transplant rejection mediated by recipient myeloid cells and other SIRPα-positive immune cells. It neither transduces nor essentially transmits signals of inhibitory effects on cell survival, and it neither inhibits nor essentially inhibits angiogenesis, thereby reducing transplant injury and cellular immunogenicity.

According to a preferred embodiment, the CD47 mutant is a human CD47 mutant possessing an amino acid sequence as set forth in SEQ ID NO.1.

According to a preferred embodiment, the signal peptide sequence (1-18aa) of human CD47 is as set forth in SEQ ID NO.15.

According to a preferred embodiment, the IgV domain sequence (19-137aa) of the human CD47 mutant is as set forth in SEQ ID NO.17.

According to a preferred embodiment, the CD47 mutant is a mouse CD47 mutant possessing an amino acid sequence as set forth in SEQ ID NO.2.

According to a preferred embodiment, the signal peptide sequence (1-18aa) of mouse CD47 is as set forth in SEQ ID NO.19.

According to a preferred embodiment, the IgV domain sequence (19-161aa) of the mouse CD47 mutant is as set forth in SEQ ID NO.21.

The second aspect of the present disclosure provides a humanized animal that transgenically expresses any one of the human CD47 mutants provided in the first aspect of the present disclosure.

According to a preferred embodiment, the humanized animal is a pig and/or a non-human primate.

According to a preferred embodiment, the non-human primate is a monkey, ape, or baboon.

The third aspect of the present disclosure provides applications of the aforementioned CD47 mutant in production and preparation of hypoimmunogenic allogeneic transgenic cell grafts or hypoimmunogenic allogeneic cell therapy products.

According to a preferred embodiment, the hypoimmunogenic allogeneic transgenic cell grafts or hypoimmunogenic allogeneic cell therapy products are selected from a group consisting of hematopoietic stem cells (HSCs), islet cells, embryonic stem cells (ESCs), induced pluripotent stem cells (iPSCs), and stem cell-differentiated derivatives.

The fourth aspect of the present disclosure provides a method for producing hypoimmunogenic allogeneic transgenic cell grafts or hypoimmunogenic allogeneic cell therapy products, the method comprising: transgenically overexpressing the CD47 mutant protein provided in the first aspect of the present disclosure in an allogeneic cell population; obtaining and culturing the mutant-overexpressing cells to expand the cell population; and harvesting the expanded mutant-overexpressing cells as the grafts or therapeutic products. The use of the obtained mutant-overexpressing cells as grafts or therapeutic products solves the problem of difficult or lengthy acquisition of autologous cells. Overexpressing the CD47 mutant in allogeneic cells can amplify the strength of the immunosuppressive signals sent to the recipient's immune system, avoid phagocytosis and rejection by myeloid cells and other SIRPα-positive immune cells, and further reduce the immunogenicity of the cells.

Preferably, the method for producing hypoimmunogenic allogeneic transgenic cell grafts or hypoimmunogenic allogeneic cell therapy products comprises: introducing the CD47 mutant provided in the first aspect of the present disclosure into the allogeneic cell population to achieve high expression; culturing the cells to expand the cell population; and harvesting the cells.

Preferably, the method for producing hypoimmunogenic allogeneic transgenic cell grafts or hypoimmunogenic allogeneic cell therapy products comprises: introducing the coding sequence of the CD47 mutant into the allogeneic cell population using existing mature transgenic techniques, such as site-specific recombination or transposon insertion methods, to achieve high expression; culturing the cells to expand the cell population; and harvesting the cells.

High expression of the CD47 mutant can amplify the strength of the immunosuppressive signals sent to the recipient's immune system and reduce the immunogenicity of the cells.

The fifth aspect of the present disclosure provides hypoimmunogenic allogeneic transgenic cell grafts or hypoimmunogenic allogeneic cell therapy products produced or prepared by the aforementioned method for producing hypoimmunogenic allogeneic transgenic cell grafts or hypoimmunogenic allogeneic cell therapy products. Overexpression of the CD47 mutant significantly reduces the immunogenicity of allogeneic cells without inhibiting cell survival, thereby reducing the host immune system's attack and rejection of the graft and cell therapy products, and improving the survival rate and functionality of the transgenic cell grafts and cell therapy products.

The sixth aspect of the present disclosure provides a method for treating a disease or condition in a subject, comprising: administering to the subject the hypoimmunogenic allogeneic transgenic cell grafts or hypoimmunogenic allogeneic cell therapy products provided in the fifth aspect of the present disclosure. Such method, by overexpressing the CD47 mutant in allogeneic cells, significantly reduces the host's immune rejection response to the grafts or cell therapy products.

The seventh aspect of the present disclosure provides a composition comprising the CD47 mutant protein provided in the first aspect of the present disclosure and a pharmaceutically acceptable carrier. Utilizing the properties of the CD47 mutant protein, in combination with an appropriate carrier, to achieve more effective drug delivery and therapeutic effects. Acceptable carriers are those that not only protect the CD47 mutant protein from external environmental influences but also help it cross biological barriers to reach target tissues or cells.

The eighth aspect of the present disclosure provides a method for treating a disease or condition in a subject, comprising: administering to the subject the composition provided in the seventh aspect of the present disclosure. This composition enhances the efficiency of drug delivery and the effectiveness and safety of treatment.

The ninth aspect of the present disclosure provides a nucleic acid molecule encoding the CD47 mutant provided in the first aspect of the present disclosure. The nucleic acid sequence encodes a mutated form of the CD47 protein, which, by altering the structure and function of the CD47 protein, reduces treatment-related side effects.

The tenth aspect of the present disclosure provides a composition comprising the nucleic acid molecule encoding the CD47 mutant provided in the ninth aspect of the present disclosure and a pharmaceutically acceptable carrier. By using the composition provided in the present disclosure, the delivery efficiency of the nucleic acid molecule encoding the CD47 mutant can be significantly improved, ensuring its efficient expression in target cells for precise regulation of the immune response.

The eleventh aspect of the present disclosure provides a method for producing or preparing the CD47 mutant protein provided in the first aspect of the present disclosure, comprising: introducing the nucleic acid molecule provided in the ninth aspect of the present disclosure into a host cell; and culturing the host cell to express the mutant. By this way, high-efficiency expression of the CD47 mutant can be achieved, enhancing the specificity and effectiveness of the treatment.

The technical effects of the present disclosure are as follows:

Previous reports have described CD47 mutant molecules structurally similar to those of the present disclosure but with non-identical sequences (Rebres, R A et al. 2001. J Biol Chem 276, 34607-34616; Lindberg, F P et al. 1996. J Cell Biol 134, 1313-1322). The main structure of these reported molecules also consists of the IgV domain of CD47 combined with the GPI anchor of DAF. The sequence difference between the reported molecules and the mutant of the present disclosure lies in the presence of four amino acids with a relative high tendency to be functionally active, HETT, at the junction of the IgV and GPI domains in the reported molecules, which are absent in the mutant of the present disclosure. These four amino acids include a positively charged histidine (H, basic), a negatively charged glutamic acid (E, acidic), and two threonines (T) prone to various modifications, which may introduce structural instability and unpredictable functional effects to the reported molecules. Furthermore, the reported molecules have no technical effects, as stated in the background technology of the present disclosure: “research results in the current field broadly agree that the transmembrane domain of CD47 and a key disulfide bond between the transmembrane domain and the extracellular IgV domain are essential for binding and activating the receptor SIRPα to exert CD47's beneficial anti-rejection effects.” Therefore, the existing reports have never tested the relevant functions of the reported molecules in transplantation, meaning that the current technology has not disclosed reports related to the technical effects of the present disclosure. All existing literature only uses the reported molecules as negative controls for certain transmembrane signaling (such as calcium signaling) or as positive controls for certain functions that can be exerted by the IgV domain alone (such as vitronectin bead binding).

Additionally, although the existing technology discloses binding sites for CD47 and SIRPα, with the primary binding sites located outside the intracellular and transmembrane regions of CD47, specifically on the IgV domain, it is currently recognized that the presence of a critical disulfide bond between the CD47 transmembrane region and the IgV domain is a prerequisite for CD47 to effectively bind and activate SIRPα (Rebres, R A et al. 2001. J Biol Chem 276, 34607-34616). Furthermore, according to a report in 2015, researchers engineered and expressed a mutant of CD47 IgV domain (referred to as ECD, extracellular domain, in the article) without the intracellular and transmembrane regions, retaining its binding sites with SIRPα and making modifications at other positions. This study found that the engineered soluble IgV mutant had a stronger binding ability to SIRPα (the modified soluble IgV domain mutant of CD47 could bind to SIRPα with a binding affinity much higher than that of wild-type CD47). Specifically, the study found that this IgV mutant could block the activation of SIRPα and its inhibition of the phagocytic ability of myeloid cells (Ho, C C et al. 2015. J Biol Chem 290, 12650-12663). That is, in the existing technology, the soluble IgV mutant, which exists independently of the intracellular and transmembrane regions, cannot activate SIRPα after strongly binding to SIRPα, nor can it produce the effect of inhibiting phagocytosis and macrophage-mediated transplant rejection (i.e., when the IgV mutant binds to SIRPα, it not only fails to activate SIRPα but also blocks the activation of SIRPα, preventing it from inhibiting the phagocytic ability of myeloid cells).

Supported by the research results of the aforementioned existing technology, it is currently a common understanding that for CD47 to effectively bind to and activate SIRPα, thereby inhibiting macrophage phagocytosis and transplant rejection, the transmembrane region is indispensable. Therefore, molecules similar to the mutant provided by the present disclosure, which consisting of only the functional IgV domain of CD47 without the intracellular and transmembrane domains, have never been considered for the activation of SIRPα and the inhibition of myeloid cell phagocytosis. The technical effects of the CD47 mutant provided by the present disclosure were an unexpected discovery. Surprisingly, the CD47 mutant provided by the present disclosure can still retain the ability to inhibit macrophage phagocytosis and transplant rejection in the absence of the transmembrane and intracellular regions, contrary to the existing understanding of CD47 structure and function, and achieves unexpected technical effects.

The present disclosure achieves this by removing CD47 transmembrane and intracellular regions with negative effects, the removed domains are potentially involved in transmission of signals which aggravate tissue injury by inhibitory effects on cell survival, angiogenesis, and blood flow suppression, while fusing the beneficial functional segments (extracellular IgV domain of CD47) involved in inhibiting myeloid cell phagocytosis and cytotoxic activity with the GPI membrane anchor attachment signal from the human DAF protein to obtain a CD47 mutant. Structural features of the CD47 mutant provided by the present disclosure enabled the extracellular domain of CD47 to be correctly located and highly expressed on the cell membrane, just like wild-type CD47, without dependence of the transmembrane and intracellular regions, through the fusion of lipid raft-specific GPI membrane anchor. Experimental results demonstrate that, compared with wild-type CD47, the CD47 mutant provided by the present disclosure no longer mediate CD47-specific apoptosis and no longer inhibit angiogenesis. In existing research cognition, it is widely recognized that the transmembrane region of CD47 is essential for the inhibition of transplant rejection by CD47. However, the CD47 mutant provided by the present disclosure can still retain the ability to inhibit macrophage phagocytosis and transplant rejection in the absence of the transmembrane and intracellular regions. The present disclosure, through in vivo and in vitro experimental results, confirms that the CD47 mutant can provide cells with equivalent resistance to macrophage phagocytosis and transplant rejection as wild-type CD47 in the absence of the transmembrane and intracellular regions, while not affecting normal cell functions. Thus, the CD47 mutant successfully retains the beneficial function such as anti-rejection and eliminates the negative effects of wild-type CD47 such as aggravating graft injury.

Therefore, the utilization of the CD47 mutant provided by the present disclosure in improvement and breeding of transgenic donor animals for xenotransplantation is conducive to increasing the survival rate of xenografts and protecting their functions, which is highly significant for advancing clinical application of xenotransplantation. Additionally, based on the beneficial effects mentioned above, the CD47 mutant is also suitable for optimizing the preparation schemes of hypoimmunogenic transgenic cell grafts, and it also has translational value for the manufacturing of hypoimmunogenic allogeneic cell therapy products.

### BRIEF DESCRIPTION OF THE DRAWINGS

FIG. 1 illustrates schematic diagram on topological structures of human/mouse CD47 mutants and wild-type controls, along with the construction of their lentiviral expression plasmids.

FIG. 2 shows colony PCR product analysis results of positive clones which contain the lentiviral plasmids with DNA inserts encoding human/mouse CD47 mutants and wild-type controls.

FIGS. 3A-E depict expression levels of human/mouse CD47 mutants and wild-type controls in CD47KO Jurkat/A20/PK15/EA.hy926, or wild-type A20 cell lines. Specifically, FIG. 3A shows the surface and total (surface plus intracellular) staining results using anti-human CD47 antibody of CD47KO Jurkat cells stably expressing human CD47 mutant or wild-type control, compared with that of CD47KO Jurkat cells. FIG. 3B presents the surface staining results using anti-mouse CD47 antibody of CD47KO A20 cells stably expressing mouse CD47 mutant or wild-type control, compared with that of CD47KO and wild-type A20 (WT) cells. FIG. 3C shows the surface staining results using anti-mouse CD47 antibody of wild-type A20 cells stably expressing mouse CD47 mutant or wild-type control, compared with that of CD47KO and wild-type A20 (WT) cells. FIG. 3D depicts the surface staining results using anti-human CD47 antibody of CD47KO PK15 cells stably expressing human CD47 mutant (hCD47-IgV), compared with that of CD47KO PK15 cells. FIG. 3E shows the surface staining results using anti-human CD47 antibody of CD47KO EA.hy926 cells stably expressing human CD47 mutant or wild-type control, compared with that of CD47KO and wild-type EA.hy926 (WT) cells.

FIG. 4 compares apoptosis induction in CD47KO Jurkat cells by the human CD47 mutant and wild-type control under ligation of CD47-specific pro-apoptotic antibody CC2C6 (** P<0.01; ns indicates no significance).

FIGS. 5A-G present in vitro and in vivo comparison of protective effects of human CD47 mutant and wild-type control against macrophage phagocytosis of CD47KO Jurkat cells. FIGS. 5A-C show protections against phagocytosis of (CFSE-labeled) CD47KO Jurkat cells by transgenic expression of human CD47 mutant or wild-type control in assessment systems of macrophages derived from human PBMCs (FIG. 5A) or from bone marrow of NCG mice whose SIRPα capable of cross-reacting with human CD47 (FIG. 5B, FIG. 5C), assessed using flow cytometry (FIGS. 5A and 5B) and confocal imaging (FIG. 5C). FIGS. 5D-F depict a correlation between the expression levels of human CD47 mutant and wild-type control and their protective effects against phagocytosis in an assessment system of macrophages from NCG mice whose SIRPα capable of cross-reacting with human CD47. Specifically, FIG. 5D shows different expression levels (MFI) of human CD47 mutant and wild-type control in CD47KO Jurkat cells (high expression group: hCD47-IgV^{hi}, hCD47-iso2^{hi}; low expression group: hCD47-IgV^{lo}, hCD47-iso2′°). FIG. 5E presents phagocytosis by NCG mouse macrophages corresponding to target cell groups with different CD47 expression levels in FIG. 5D assessed using flow cytometry analysis. FIG. 5F is a statistical graph of phagocytosis levels in FIG. 5E (****P<0.0001; *P<0.05). FIG. 5G shows the comparison of the abilities of human CD47 mutant and wild-type control protecting CD47KO Jurkat cells against phagocytosis and rejection (in vivo) in a leukemia model established with Jurkat cells in NCG mice whose SIRPα capable of cross-reacting with human CD47, assessed by survival curves of the mice (*P<0.05; **P<0.01; ns indicates no significance).

FIGS. 6A-E demonstrate the anti-phagocytic effects of CD47 mutant of a different species from the human mutant of the present disclosure, using both in vitro and in vivo experiments. Specifically, the protective ability of mouse CD47 mutant, structurally corresponding to the human mutant provided by the present disclosure, against macrophage phagocytosis of CD47KO A20 cells is assessed using mouse wild-type CD47 as a control. Specifically, FIG. 6A shows analysis of in vitro anti-phagocytic effects provided by mouse CD47 mutant and the wild-type control for CD47KO A20 cells (CFSE labeled) in an assessment system of macrophages derived from BALB/c mice bone marrow (F4/80-APC stained) by flow cytometry (left panel: representative flow cytometry profiles; right panel: statistical graph, ***P<0.001, ****P<0.0001, ns indicates no significance). FIG. 6B presents corresponding confocal imaging results of FIG. 6A. FIG. 6C shows the comparison of in vivo protections against phagocytosis and rejection provided by mouse CD47 mutant and the wild-type control for CD47KO A20 cells which were injected into female BALB/c mice (3.5Gy irradiated) through the tail veins to established a leukemia mouse model, assessed by survival curves (*P<0.05; **P<0.01; ns indicates no significance). FIG. 6D shows survival curves of female (left) or male (right) Rag^{-/-} BALB/c mice injected through the tail veins with A20 cells to established a leukemia mouse model, comparing the in vivo protections against phagocytosis and rejection provided by mouse CD47 mutant and the wild-type control for CD47KO A20 cells (*P<0.05; ns indicates no significance). FIG. 6E presents a comparison of enhanced in vivo anti-rejection capabilities provided by mouse CD47 mutant and the wild-type control for wild-type A20 cells in a leukemia model established by tail vein injection of A20 cells into BALB/c mice (3.5Gy irradiated), assessed by survival curves (*P<0.05; ***P<0.001; ns indicates no significance).

FIGS. 7A-E illustrates comparison of engraftment and differentiation of hematopoietic stem/progenitor cells (LSK HSPCs) from GFP-transgenic mice with endogenous CD47 knocked out (GFP⁺ CD47KO C57BL/6), stably expressing mouse CD47 mutant or the wild-type control, in syngeneic recipient mice (GFP-negative WT C57BL/6). Specifically, FIG. 7A shows chimerism and lineage distribution of GFP-positive donor cells in the peripheral blood cells of recipient mice at indicated post-transplant time points. FIGS. 7B-D depict chimerism percentages of different cell lineages derived from donor HSPCs (GFP⁺) in the spleen (7B), lymph nodes (7C), and bone marrow (7D) when recipient mice were sacrificed 14 weeks after transplantation. FIG. 7E presents percentages of GFP-positive donor-derived LSK, GMP, CMP, MEP, and CLP cells in the bone marrow of recipient mice from both experimental groups.

FIG. 8 demonstrates protective ability of transgenic expression of the human CD47 mutant of the present disclosure against phagocytosis of CD47KO porcine epithelial PK15 cells by human macrophages. The protection effect against phagocytosis of CD47KO PK15 cells provided by transgenic expression of the human CD47 mutant is evaluated in an assessment system of human PBMC-derived macrophages at indicated time points post co-culture of effector (macrophage) and target (porcine PK15) cells (*P<0.05; **P<0.01).

FIGS. 9A-D presents the results of in vitro and in vivo angiogenesis assays of CD47KO EA.hy926 endothelial cells (ECs) stably expressing the human CD47 mutant of the present disclosure or wild-type control, compared with that of CD47KO and WT EA.hy926 cells. Specifically, FIG. 9A shows the results of an in vitro tube formation assay, with representative images of tube formation on the left and statistical graphs of EC junction numbers and total length in three randomly selected fields analyzed using ImageJ software with the Angiogenesis Analyzer plugin on the right (*P<0.05; **P<0.01; ns indicates no significance). FIG. 9B displays the results of in vitro angiogenesis assays at passages 3 (P-3) and passages 10 (P-10) post-thaw of the endothelial cells, with representative images of tube formation on the left and statistical graphs of EC junction number and total length in three randomly selected fields on the right (*P<0.05; **P<0.01; ns indicates no significance). FIG. 9C depicts H&E staining (upper panel) and anti-human CD31 immunohistochemical staining results (lower panel) of neovascularization in Matrigel plugs separately containing different groups of ECs and subcutaneously implanted into NCG mice, with arrows pointing to typical neovessels. FIG. 9D shows photograph (left panel), H&E staining (middle panel), and anti-human CD31 immunohistochemical staining results (right panel) of blank Matrigel plugs without endothelial cells, dissected out on day 10 post-implantation from NCG mice.

### DETAILED DESCRIPTION OF THE APPLICATION

The following detailed explanation is provided in conjunction with the accompanying figures.

The ensuing embodiments are solely intended to further elucidate the beneficial effects of the CD47 mutant in the present disclosure and should not be construed to limit its scope or applicability. Based on the experimental results of the embodiments, it is reasonably inferred that other CD47 mutants possessing the main structural characteristics of the CD47 mutants described herein can similarly achieve the present disclosure's objectives of circumventing the detrimental effects of wild-type CD47 while retaining its beneficial functions such as inhibiting phagocytosis and transplant rejection to varying extents. Specifically, any CD47 recombinant protein obtained by fusing any GPI attachment signal sequences that enable the correct transport and localization of CD47 to the cell membrane, and its enrichment in lipid rafts, to the C-terminus of any CD47 IgV domain, including its mutants and truncated forms, that can effectively recognize and bind to SIRPα, may potentially achieve the beneficial effects of the mutants described in the present disclosure. These reasonable inferences can be applied to the further design and optimization of new CD47 mutants with structural characteristics of the mutants described in the present disclosure, enhancing their beneficial effects.

For those skilled in the art, the specific meanings of the terms used in the present disclosure can be understood based on the given context. The experimental procedures described in the following examples are standard unless otherwise specified. Reagents used in the following examples, unless otherwise noted, are commercially available.

Main sources of reagents for the present disclosure are as follows:

Plasmid pSpCas9(BB)-2A-GFP (PX458): Catalog #48138, a gift from the Feng Zhang's Lab at Addgene; Asc I and Sal I enzymes: purchased from ThermoFisher, USA; BD Pharmingen™ Alexa Fluor®647 mouse anti-human CD47 antibody: clone B6H12, from BD Bioscience, USA, Cat #561249; Alexa Fluor®647 Anti-mouse CD47 antibody and PE anti-mouse CD47 antibody: both clone miap301, from Biolegend, USA; anti-human CD47 (unlabeled primary antibody), which cross-reacts with porcine CD47: clone BRIC126, from Bio-Rad, USA; FITC goat anti-mouse IgG antibody (fluorescently labeled secondary antibody, minimal x-reactivity): clone Poly4053, from Biolegend, USA; APC Anti-human CD172α/β (SIRPα/β)-antibody: clone SE5A5, from Biolegend, USA; APC Anti-mouse F4/80 antibody: clone BM8, from Biolegend, USA; purified anti-human CD47 antibody inducing CD47-dependent cell death: clone CC2C6, from Biolegend, USA, Cat #323102; APC Annexin V: from Biolegend, USA; BD Horizon™ BV711 anti-mouse CD11b antibody: clone M1/70, from BD Bioscience, USA; PE anti-mouse CD11b Monoclonal antibody: clone M1/70, from eBioscience, USA; Anti-human CD31 polyclonal antibody: from Abcam, UK; Recombinant human macrophage colony-stimulating factor (M-CSF) protein: from R&D Systems, USA; Recombinant mouse M-CSF protein: from Biolegend, USA; Lipopolysaccharide (LPS): from Sigma, Germany; CFSE: from ThermoFisher, USA; PKH67 Green Fluorescent Cell Linker Kit: from Sigma-Aldrich, Germany; Matrigel Matrix: from Corning Life Sciences, USA; 293T cells, Jurkat and A20 tumor cells: from ATCC (American Type Culture Collection), USA; PK15 cells: from National Infrastructure of Cell Line Resource, CHINA; EA.hy926 cells: from National Collection of Authenticated Cell Cultures, CHINA. BALB/cAnNCrl and C57BL/6Jnifdc mice: Beijing Vital River Laboratory Animal Technology company, CHINA. NOD/ShiLtJGpt Prkdc^{em26cd52}Il2rg^{em26Cd22}/Gpt (NCG) mice: Gempharmatech, CHINA. Rag^{-/-} BALB/c mice: Biogle GeneTech, CHINA. In addition to the primary reagents mentioned above, the antibodies of the cell surface markers used to identify mouse hematopoietic stem cells and the antibodies of the cell surface markers used to identify mouse leukocytes, myeloid cells, T cells, B cells, and other cell lineages in these embodiments were all purchased from BioLegend, USA.

The sequences involved in the present disclosure are as follows:
Amino acid sequence of human CD47 mutant (hCD47-IgV) (SEQ ID NO.1, MWPLVAALLLGSACCGSAQLLFNKTKSVEFTFCNDTVVIPCFVTNMEAQNTTEVYVKW KFKGRDIYTFDGALNKSTVPTDFSSAKIEVSQLLKGDASLKMDKSDAVSHTGNYTCEVT ELTREGETIIELKYRVVSWFPNKGSGTTSGTTRLLSGHTCFTLTGLLGTLVTMGLLT); Amino acid sequence of mouse CD47 mutant (mCD47-IgV) (SEQ ID NO.2, MWPLAAALLLGSCCCGSAQLLFSNVNSIEFTSCNETVVIPCIVRNVEAQSTEEMFVKWK LNKSYIFIYDGNKNSTTTDQNFTSAKISVSDLINGIASLKMDKRDAMVGNYTCEVTELSR EGKTVIELKNRTAFNTDQGSACSYEEEKGGCKLVSWFSPNEKPNKGSGTTSGTTRLLSGH TCFTLTGLLGTLVTMGLLT); Amino acid sequence of human wild-type CD47 (hCD47-iso2) (SEQ ID NO.3, MWPLVAALLLGSACCGSAQLLFNKTKSVEFTFCNDTVVIPCFVTNMEAQNTTEVYVKW KFKGRDIYTFDGALNKSTVPTDFSSAKIEVSQLLKGDASLKMDKSDAVSHTGNYTCEVT ELTREGETIIELKYRVVSWFSPNENILIVIFPIFAILLFWGQFGIKTLKYRSGGMDEKTIALL VAGLVITVIVIVGAILFVPGEYSLKNATGLGLIVTSTGILILLHYYVFSTAIGLTSFVIAILVIQ VIAYILAVVGLSLCIAACIPMHGPLLISGLSILALAQLLGLVYMKFVASNQKTIQPPRNN); Amino acid sequence of mouse wild-type CD47 (mCD47-iso2) (SEQ ID NO.4, MWPLAAALLLGSCCCGSAQLLFSNVNSIEFTSCNETVVIPCIVRNVEAQSTEEMFVKWK LNKSYIFIYDGNKNSTTTDQNFTSAKISVSDLINGIASLKMDKRDAMVGNYTCEVTELSR EGKTVIELKNRTAFNTDQGSACSYEEEKGGCKLVSWFSPNEKILIVIFPILAILLFWGKFGI LTLKYKSSHTNKRIILLLVAGLVLTVIVVVGAILLIPGEKPVKNASGLGLIVISTGILILLQY NVFMTAFGMTSFTIAILITQVLGYVLALVGLCLCIMACEPVHGPLLISGLGIIALAELLGLV YMKFVASNQRTIQPPRNR); Amino acid sequence of human CD47 extracellular domain with transmembrane and intracellular regions removed (including signal peptide and IgV domain, 1-137aa, SEQ ID NO.5, MWPLVAALLLGSACCGSAQLLFNKTKSVEFTFCNDTVVIPCFVTNMEAQNTTEVYVKW KFKGRDIYTFDGALNKSTVPTDFSSAKIEVSQLLKGDASLKMDKSDAVSHTGNYTCEVT ELTREGETIIELKYRVVSWF); Amino acid sequence of mouse CD47 extracellular domain with a deletion of the transmembrane and intracellular regions (including signal peptide and IgV domain, 1-161aa, SEQ ID NO.6, MWPLAAALLLGSCCCGSAQLLFSNVNSIEFTSCNETVVIPCIVRNVEAQSTEEMFVKWK LNKSYIFIYDGNKNSTTTDQNFTSAKISVSDLINGIASLKMDKRDAMVGNYTCEVTELSR EGKTVIELKNRTAFNTDQGSACSYEEEKGGCKLVSWFSPNEK); Amino acid sequence of glycosylphosphatidylinositol (GPI) attachment signal (SEQ ID NO.7, human, PNKGSGTTSGTTRLLSGHTCFTLTGLLGTLVTMGLLT); Coding sequence of human CD47 mutant (SEQ ID NO.8); Coding sequence of mouse CD47 mutant (SEQ ID NO.9); Coding sequence of GPI attachment signal (SEQ ID NO.10, human); Coding sequence of human extracellular domain (including signal peptide and IgV domain) (SEQ ID NO.11); Coding sequence of mouse extracellular domain (including signal peptide and IgV domain) (SEQ ID NO.12); Coding sequence of human wild-type CD47 (SEQ ID NO.13); Coding sequence of mouse wild-type CD47 (SEQ ID NO.14); Signal peptide sequence of human CD47 (1-18aa, SEQ ID NO.15, MWPLVAALLLGSACCGSA); Coding sequence of human CD47 signal peptide (SEQ ID NO.16); Amino acid sequence of human CD47 IgV domain (19-137aa, SEQ ID NO.17, QLLFNKTKSVEFTFCNDTVVIPCFVTNMEAQNTTEVYVKWKFKGRDIYTFDGALNKSTV PTDFSSAKIEVSQLLKGDASLKMDKSDAVSHTGNYTCEVTELTREGETIIELKYRVVSWF); Coding sequence of human CD47 IgV domain (SEQ ID NO.18); Signal peptide sequence of mouse CD47 (1-18aa, SEQ ID NO.19, MWPLAAALLLGSCCCGSA); Coding sequence of mouse CD47 signal peptide (SEQ ID NO.20); Amino acid sequence of mouse CD47 IgV domain (19-161aa, SEQ ID NO.21, QLLFSNVNSIEFTSCNETVVIPCIVRNVEAQSTEEMFVKWKLNKSYIFIYDGNKNSTTTDQ NFTSAKISVSDLINGIASLKMDKRDAMVGNYTCEVTELSREGKTVIELKNRTAFNTDQGS ACSYEEEKGGCKLVSWFSPNEK); Coding sequence of mouse CD47 IgV domain (SEQ ID NO.22); Sequencing results of human CD47 mutant (hCD47-IgV) recombinant lentiviral recombinant plasmid (SEQ ID NO.23); Sequencing results of human wild-type CD47 (hCD47-iso2) recombinant lentiviral plasmid (SEQ ID NO.24); Sequencing result of mouse CD47 mutant (mCD47-IgV) recombinant lentiviral plasmid (SEQ ID NO.25); Sequencing result of mouse wild-type CD47 (mCD47-iso2) recombinant lentiviral plasmid (SEQ ID NO.26).

Through a series of in vivo and in vitro experimental validations, the present disclosure confirms that, compared with wild-type CD47, the obtained CD47 mutant eliminates the detrimental effects of inhibiting cell survival and angiogenesis while retaining the beneficial functions such as the inhibition of transplant rejection. Additionally, such mutant effectively protects porcine cells from human macrophage phagocytosis without affecting the functionality of cell products, such as hematopoietic stem cells. Therefore, the CD47 mutant provided by the present disclosure has significant translational values for optimizing breeding schemes of transgenic donor animals for xenotransplantation (e.g., pigs, and non-human primates such as monkeys, apes, or baboons) and for preparing hypoimmunogenic allogeneic transgenic cell grafts and therapy products (including but not limited to embryonic stem cells, induced pluripotent stem cells, hematopoietic stem cells, stem cell derivatives, and islet cells). This CD47 mutant is expected to enhance the survival rate and functionality, and reduce the risk of transplant injury and rejection in both transgenic donor animal cells and tissues, as well as in transgenic allogeneic cell grafts. The coding sequence (CDS) of the CD47 mutant can be directly obtained by chemical synthesis and purchased commercially or amplified using polymerase chain reaction (PCR) technology. Future applications of this CD47 mutant in breeding transgenic animals or producing hypoimmunogenic transgenic cell products can be achieved through existing and mature genome editing techniques such as site-specific insertion or transposon-mediated insertion.

### Embodiment 1

This embodiment involves the stable expression of human/mouse CD47 mutants or human/mouse wild-type CD47 in Jurkat, EA.hy926, A20, and PK15 cell lines with endogenous CD47 knockout, as well as in wild-type A20 cell line.

1. Establishment of Human Jurkat, Human EA.hy926, Mouse A20, and Porcine PK15 Cell Lines with Endogenous CD47 Knockout

Endogenous CD47 in human Jurkat, human EA.hy926, mouse A20, and porcine PK15 cells was knocked out using existing CRISPR/Cas9 gene-editing technology to eliminate interference from endogenous CD47 during the functional testing of human/mouse CD47 mutants. Cells with endogenous CD47 knockout are denoted as CD47KO.

The specific steps for knocking out endogenous CD47 in these cell lines are as follows: Transfect Jurkat and EA.hy926 cells with the pSpCas9(BB)-2A-GFP (PX458) plasmid containing human CD47-specific sgRNA (CTACTGAAGTATACGTAAAG, SEQ ID NO.28) according to the Lipofectamine 2000 transfection protocol provided by the manufacturer; transfect A20 cells with the pSpCas9(BB)-2A-GFP (PX458) plasmid containing mouse Cd47-specific sgRNA (TTGGCGGCGGCGCTGTTGCT, SEQ ID NO.29); transfect porcine PK15 cells with the pSpCas9(BB)-2A-GFP (PX458) plasmid containing porcine CD47-specific sgRNA (AACGGTGAATTCTACAGATT, SEQ ID NO.30). 48 hours after transfection, successfully transfected GFP-positive cells were sorted and collected by flow cytometry for further culture.

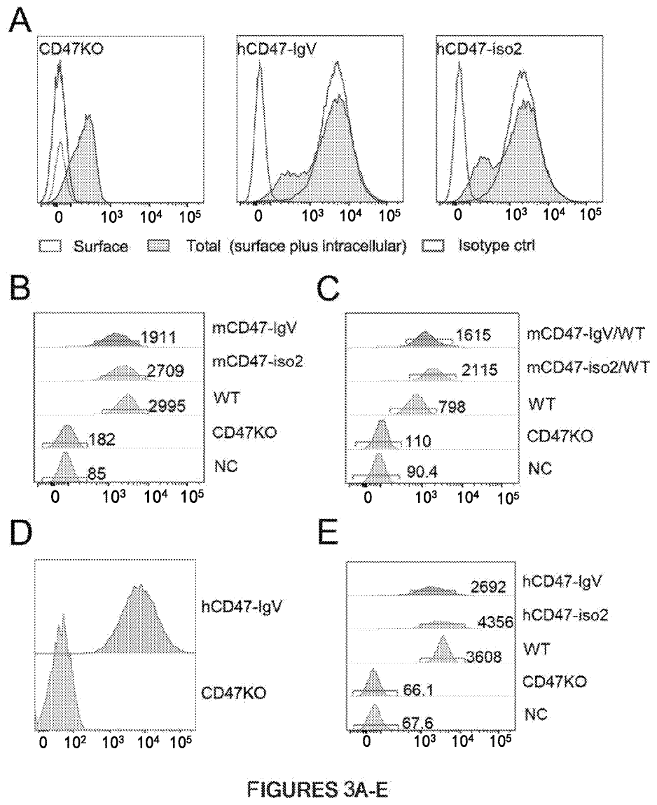

The sorted cells were cultured for one week, then Jurkat cells and EA.hy926 cells were stained using AF647-labeled anti-human CD47 antibody (B6H12), while A20 cells were stained using AF647-labeled anti-mouse CD47 antibody (MIAP301) and PK15 cells were sequentially stained using unlabeled primary antibody (BRIC126) that can bind to porcine CD47, followed by a fluorescently labeled secondary antibody (FITC goat anti-mouse IgG antibody), aforementioned stained cells were sorted again to obtain CD47-negative cells. Through three rounds of negative cell sorting, CD47KO Jurkat, CD47KO EA.hy926, CD47KO A20, and CD47KO PK15 cell lines with endogenous CD47 knockout were successfully established. CD47 knockout results analyzed by flow cytometry are shown in FIG. 3. Specifically, the flow cytometry results for CD47 expression of each CD47KO cell line are detailed in FIGS. 3A-3E.

2. Construction of Human/Mouse Mutant/Wild-Type CD47 pRRLsin Recombinant Lentiviral Plasmids

Using extracted human and mouse CD47 T-vectors as templates, PCR amplification was performed to obtain wild-type CD47 coding sequence fragments flanked by Asc I and Sal I restriction sites and protective bases (human wild-type CD47 coding sequence is SEQ ID NO.13, mouse wild-type CD47 coding sequence is SEQ ID NO.14).

Similarly, using the extracted decay accelerating factor (DAF) T-vector as a template, the coding sequence of the glycosylphosphatidylinositol (GPI) attachment signal (SEQ ID NO.10) was amplified by PCR. The extracted human/mouse CD47 T-vectors were used as templates for PCR to amplify the coding sequences of the human CD47 extracellular domain (SEQ ID NO.11) and the mouse CD47 extracellular domain (SEQ ID NO.12), respectively. These fragments, along with the GPI attachment signal coding sequence separately, were then used as templates to splice the CD47 extracellular domain coding sequence with the GPI attachment signal coding sequence via overlap PCR, yielding human and mouse CD47-IgV mutant coding sequence fragments flanked by Asc I and Sal I sites and protective bases (human CD47 mutant coding sequence is SEQ ID NO.8, mouse CD47 mutant coding sequence is SEQ ID NO.9).

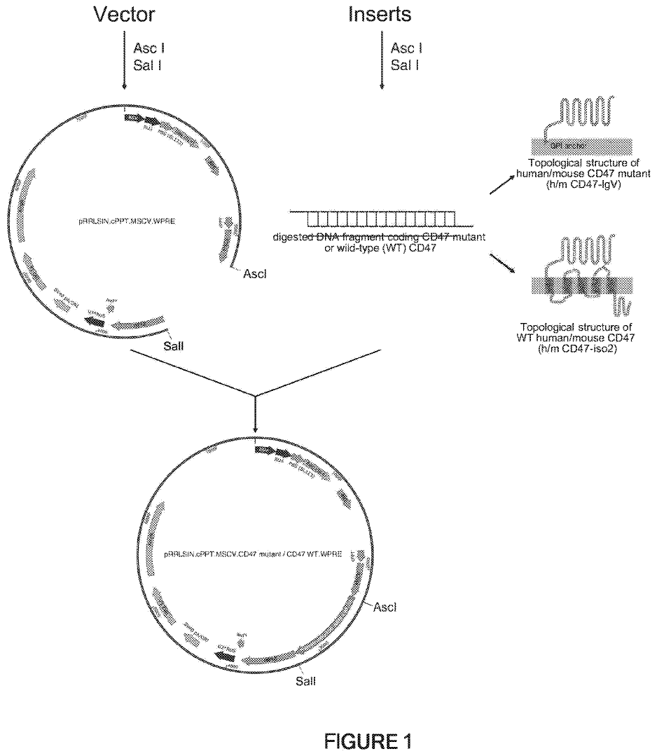

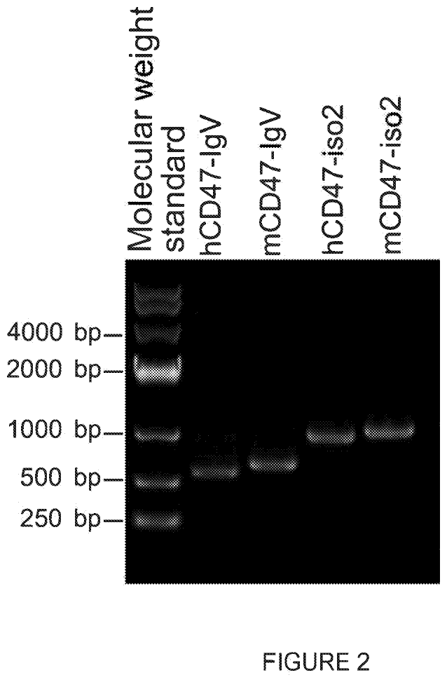

The pRRLsin lentiviral vector was double-digested with Asc I and Sal I, purified, and then ligated with PCR products similarly double-digested and purified, as depicted in FIG. 1, illustrating the successful insertion of mutant or wild-type CD47 coding sequence fragments into the pRRLsin lentiviral vector. The ligation product was transformed into Stb13 competent cells, and positive clones whose colony PCR products were of the expected sizes were selected, detailed in FIG. 2. The human CD47 mutant coding sequence fragment (denoted as hCD47-IgV in FIG. 2) was approximately 525 bp in length, the mouse CD47 mutant coding sequence fragment (mCD47-IgV) was about 597 bp in length, the human wild-type CD47 coding sequence fragment (hCD47-iso2) was approximately 918 bp in length, and the mouse wild-type CD47 coding sequence fragment (mCD47-iso2) was about 975 bp in length. The DNA molecular weight standard control used was the DL10,000 DNA Marker from TaKaRa, Japan. The insert sequences in the positive clones were sequenced by Comate Bioscience Co. Ltd., Jilin, CHINA. The sequencing results for hCD47-IgV are shown as SEQ ID NO.23; hCD47-iso2 as SEQ ID NO.24; mCD47-IgV as SEQ ID NO.25; and mCD47-iso2 as SEQ ID NO.26. The sequencing results confirm that the coding sequences inserted in the recombinant plasmids are consistent with the reference sequences.

3. Production and Transduction of Lentiviruses expressing Human/Mouse Mutant/Wild-Type CD47

A four-plasmid system, comprising the lentiviral transfer vector and three packaging plasmids, was used to transfect 293T cells cultured to 90% confluency in 10 cm dishes. Lipofectamine 2000 was used as the transfection reagent.

The amounts of plasmid DNA for transfection per dish are as follows: 9 μg pRRLsin lentiviral vector, 3 μg pMD2.G, 6 μg pMDLg/pRRE, and 6 μg pRSV-Rev.

At 72 hours after transfection, collect the supernatants, filter them through 0.45 m filters to remove cells and debris, and centrifuge at 50,000 g for 2.5 hours to concentrate the viruses. Infect human CD47KO Jurkat cells with lentivirus expressing human CD47 wild-type or CD47-IgV mutant (MOI=10) in RPMI1640 complete medium supplemented with 5 μg/ml polybrene. Similarly, infect human CD47KO EA.hy926 cells with the same lentiviruses (MOI=10) in DMEM complete medium with 6 μg/ml polybrene. Infect mouse CD47KO A20 and wild-type A20 cells with lentivirus expressing mouse CD47 wild-type or CD47-IgV mutant (MOI=10) in RPMI1640 complete medium with 5 μg/ml polybrene. Additionally, infect porcine CD47KO PK15 cells with human CD47-IgV mutant-expressing lentivirus (MOI=10) in DMEM complete medium with 6 μg/ml polybrene.

### 4. Establishment of Stable Human/Mouse Mutant/Wild-Type CD47 Expressing Cell Lines

At 72 hours after lentivirus infection, the infected CD47KO Jurkat, CD47KO EA.hy926, and CD47KO PK15 cells were stained with AF647-labeled anti-human CD47 antibody (B6H12), while the infected CD47KO A20 and wild-type A20 cells were stained with AF647-labeled anti-mouse CD47 antibody (MIAP301). Successfully infected cells expressing human or mouse CD47 were sorted and collected by flow cytometry for further culture.

The sorted cells were cultured for one week and then subjected to a second round of staining and sorting procedures for selection of CD47-positive cells. Through these two rounds of positive selection, stable cell lines expressing human/mouse wild-type or mutant CD47 were established based on the CD47KO Jurkat, CD47KO EA.hy926, CD47KO A20, and wild-type A20 cell lines, separately, and CD47KO PK15 cell line stably expressing human CD47 mutant was also established. The flow cytometry results for CD47 expression in these stable cell lines are shown in FIG. 3. FIG. 3A shows the surface and total (surface plus intracellular) staining results using anti-human CD47 antibody of CD47KO Jurkat cells stably expressing human CD47 mutant or wild-type control, compared with that of CD47KO Jurkat cells. FIG. 3B presents the surface staining results using anti-mouse CD47 antibody of CD47KO A20 cells stably expressing mouse CD47 mutant or wild-type control, compared with that of CD47KO and wild-type A20 (WT) cells. FIG. 3C shows the surface staining results using anti-mouse CD47 antibody of wild-type A20 cells stably expressing mouse CD47 mutant or wild-type control, compared with that of CD47KO and wild-type A20 (WT) cells. FIG. 3D depicts the surface staining results using anti-human CD47 antibody of CD47KO PK15 cells stably expressing human CD47 mutant (hCD47-IgV), compared with that of CD47KO PK15 cells. FIG. 3E shows the surface staining results using anti-human CD47 antibody of CD47KO EA.hy926 cells stably expressing human CD47 mutant or wild-type control, compared with that of CD47KO and wild-type EA.hy926 (WT) cells. These results in FIG. 3 indicate that both CD47 mutants and wild-type controls exhibit stable high expression on the surface of CD47KO and WT cells.

### Embodiment 2

This embodiment investigates apoptosis in CD47KO Jurkat cells stably expressing human CD47 mutant (hCD47-IgV) or human wild-type CD47 (hCD47-iso2) under CD47 ligation conditions.

The procedure involves centrifuging and washing CD47KO Jurkat cells, and CD47KO Jurkat cells stably expressing the human CD47 mutant (hCD47-IgV) or wild-type CD47 (hCD47-iso2) with PBS (Phosphate-buffered saline), then resuspending them in RPMI 1640 complete medium without antibiotics. Cells were plated at 1×10⁵ cells per well in 24-well plates and incubated at 37 °C, 5% CO₂ for 2 hours with or without 250 ng/ml CD47-specific pro-apoptotic antibody (CC2C6). Following incubation, cells were washed twice with pre-chilled PBS, resuspended in 1× Annexin V binding buffer, and stained using APC-labeled Annexin V and propidium iodide (PI, final concentration 2 μg/mL) according to the Annexin V apoptosis detection kit protocol.

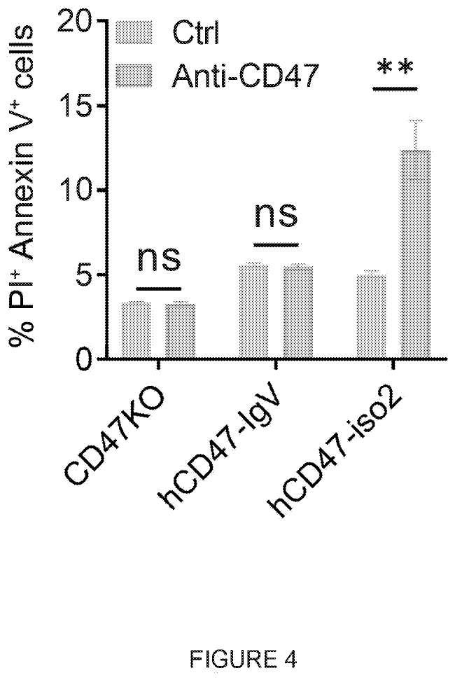

Utilizing flow cytometry, the percentages of apoptotic cells were analyzed, with results depicted in FIG. 4. The abscissa represents groups of cells stably expressing human mutant CD47 (hCD47-IgV) or wild-type CD47 (hCD47-iso2), while the ordinate indicates the percentages of apoptotic cells. Here, Ctrl denotes the untreated control group without incubation with the pro-apoptotic agonist anti-CD47 antibody (CC2C6). Anti-CD47 refers to the experimental group treated with 250 ng/ml of the pro-apoptotic agonist anti-CD47 antibody (CC2C6). The findings reveal that the human CD47 mutant (hCD47-IgV) in this embodiment does not mediate CD47-specific apoptosis in Jurkat cells, unlike the wild-type (hCD47-iso2) control which does. Specifically, under treatment with the pro-apoptotic agonist anti-CD47 antibody CC2C6, the wild-type control (hCD47-iso2) experimental group (Anti-CD47) exhibited a significantly higher percentage of apoptotic cells compared with the untreated control group (Ctrl). Conversely, the human CD47 mutant (hCD47-IgV) experimental group (Anti-CD47) showed similar results to those of CD47KO Jurkat cell (CD47 KO) experimental group (Anti-CD47), with no significant difference in apoptotic cell percentage compared to the corresponding untreated control group (Ctrl), indicating that the human mutant in this embodiment no longer mediates CD47-specific apoptosis in Jurkat cells like its wild-type counterpart, **P<0.01, ns indicates no significance.

In vitro assay results demonstrate that the CD47 mutant provided by this embodiment no longer exhibits the negative effect of the wild-type CD47 in inhibiting cell survival.

### Embodiment 3

This embodiment describes an in vitro experimental procedure investigating whether CD47 mutants protect CD47KO cells from macrophage phagocytosis.

1. In Vitro Assessment of the Ability of the Human CD47 Mutant in this Embodiment to Protect CD47KO Jurkat Cells from Phagocytosis by Macrophages Derived from Human Peripheral Blood Mononuclear Cells (PBMCs), Compared to that of Wild-Type CD47

Human PBMCs were isolated using Ficoll density gradient centrifugation and cultured for 7-9 days in RPMI 1640 complete medium supplemented with 20 ng/ml human macrophage colony-stimulating factor (M-CSF) to induce macrophage differentiation, fresh medium containing M-CSF was replaced every 2-3 days during this process. The differentiated macrophages were then seeded at 1.5×10⁵ cells per well in 24-well plates, cultured for 12 hours, and subsequently maintained in serum-free medium for an additional 2 hours.

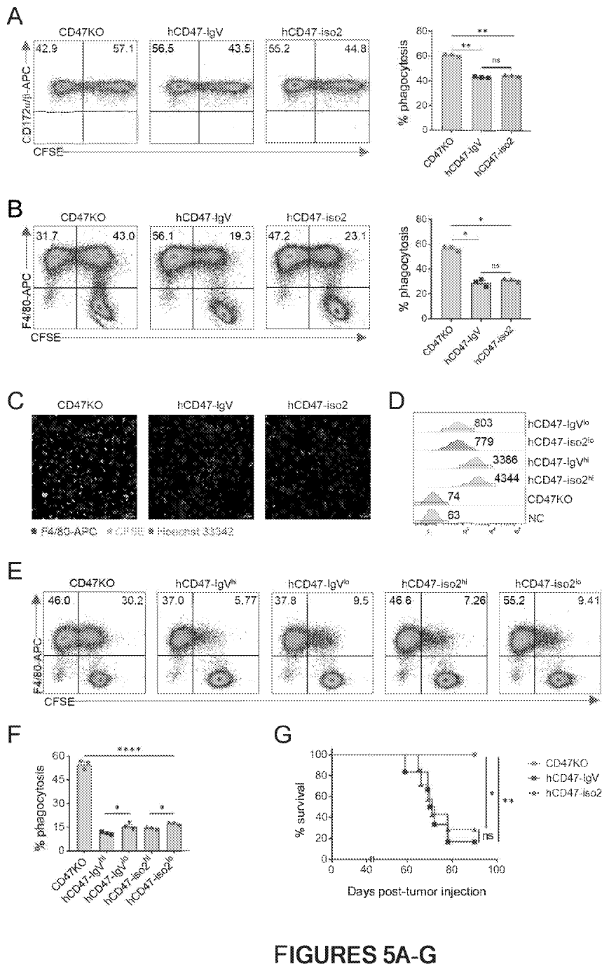

Target cells, namely 0.5 μM CFSE-labeled CD47KO Jurkat cells or CD47KO Jurkat cells stably expressing either the human CD47 mutant (hCD47-IgV) or wild-type CD47 (hCD47-iso2), were added to the wells containing the aforementioned macrophages (effector cells) at an effect-to-target ratio of 1:4. After 4 hours, the cells were harvested, stained with anti-human CD172α/β (SIRPα/β)-APC antibody, and analyzed by flow cytometry to determine the percentages of CD172α/β⁺CFSE⁺ double-positive macrophages that have phagocytosed Jurkat cells, assessing the phagocytosis of the target cells by macrophages, as shown in FIG. 5. FIG. 5A presents flow cytometry-based comparisons of the anti-phagocytic effects provided by the human mutant and wild-type CD47 for CFSE-labeled CD47KO Jurkat cells in an assessment system of human PBMC-derived macrophages. The left panel displays representative flow cytometry profiles, while the right panel shows a statistical graph of phagocytosis levels (bar graph). The x-axis of the bar graph in FIG. 5A represents the types of target cells (i.e. CD47KO, hCD47-IgV and hCD47-iso2), and the y-axis indicates the percentages of macrophages that have engulfed the target cells. FIG. 5A demonstrates that CD47KO Jurkat cells expressing hCD47-IgV and hCD47-iso2 exhibit significantly and comparably reduced phagocytosis levels compared with CD47KO Jurkat cells (denoted as CD47KO in the figure). This finding indicates that the human CD47 mutant of this embodiment has efficiency comparable to human wild-type CD47 in inhibiting phagocytosis of human macrophage in vitro.

2. In Vitro Assessment of the Ability of the Human CD47 Mutant in this Embodiment to Protect CD47KO Jurkat Cells from Phagocytosis by Bone Marrow (BM)-Derived Macrophages of NCG Mice Whose SIRPα is Capable of Cross-Reacting with Human CD47, Compared to that of Wild-Type CD47

The procedure involves obtaining bone marrow cells from 6-8-week-old NCG mice (from femur, tibia, and ilium), culturing them for 7-9 days in RPMI 1640 complete medium supplemented with 20 ng/ml mouse M-CSF, replacing the medium every 2-3 days during this process, and then activating the cells for 8-12 hours in RPMI 1640 complete medium with 20 ng/ml LPS to prepare mouse macrophages. These macrophages were seeded at 2×10⁵ cells per well in 24-well plates; for flow cytometry assays, they were directly seeded in plates, and for confocal microscopy assays, they were cultivated on microscope slides placed in the wells. After 12 hours of culturing, the macrophages were maintained in serum-free medium for an additional 2 hours. Target cells, namely 0.5 μM CFSE-labeled CD47KO Jurkat cells, or CD47KO Jurkat cells stably expressing the human CD47 mutant (hCD47-IgV) or wild-type CD47 (hCD47-iso2), were added at an effect-to-target ratio of 1:4 to the wells containing the aforementioned macrophages (effector cells). After 4 hours, for flow cytometry analysis, the cells were prepared through enzymatic digestion, and for confocal microscopy imaging, non-phagocytosed target cells were washed away with PBS, and macrophages grown on slides were fixed with 4% paraformaldehyde in PBS and stained with anti-mouse F4/80-APC antibody. Phagocytosis was assessed by flow cytometry and confocal microscopy, measuring the percentage of macrophages that had phagocytosed the target cells (F4/80⁺CFSE⁺). The results of the phagocytosis of the different target cells by macrophages are shown in FIGS. 5B and 5C. FIG. 5B presents the flow cytometry-based comparisons of the anti-phagocytic effects provided by the human CD47 mutant and wild-type control for (CFSE-labeled) CD47KO Jurkat cells in an assessment system of macrophages from the NCG mice, whose SIRPα is capable of cross-reacting with human CD47. The left panel shows the representative flow cytometry profiles and the right panel displays a statistical graph of phagocytosis levels (bar graph). FIG. 5C shows the confocal imaging-based comparisons of the protective effects of the human CD47 mutant and wild-type CD47 against phagocytosis of (CFSE-labeled) CD47KO Jurkat cells in the assessment system of macrophages derived from bone marrow of NCG mice (*P<0.05; **P<0.01; ns indicates no significance).

In the bar graph of FIG. 5B, the x-axis represents the target cell types (i.e. CD47KO, hCD47-IgV and hCD47-iso2), while the y-axis indicates the percentages of macrophages that have engulfed the target cells. Results in FIGS. 5B and 5C demonstrate that CD47KO Jurkat cells expressing hCD47-IgV and hCD47-iso2 are equally and significantly resistant to phagocytosis by bone marrow-derived macrophages of NCG mice, compared to CD47KO Jurkat cells.

FIG. 5D presents the detection results of different CD47 expression levels of CD47KO Jurkat cells expressing human CD47 mutant or wild-type CD47, categorized into high expression groups (hCD47-IgV^{hi}, hCD47-iso2 hi) and low expression groups (hCD47-IgV^{lo}, hCD47-iso2′°). FIG. 5E shows the phagocytosis levels of the target cells with different levels of CD47 expression corresponding to the cell groups in FIG. 5D by the macrophages, as analyzed by flow cytometry. FIG. 5F is the statistical graph of phagocytosis levels in FIG. 5D, where the x-axis represents the target cell types with different expression levels of human CD47 mutant and wild-type CD47, and the y-axis indicates the percentages of the macrophages that have engulfed the target cells, ****P<0.0001; *P<0.05. Results in FIGS. 5D-5F indicate that compared with that in the CD47KO group, the percentages of macrophages engulfing target cells in the hCD47-IgV^{hi}, hCD47-IgV^{lo}, hCD47-iso2^{hi} and hCD47-iso2^{lo} groups significantly decreased. Furthermore, the percentage of macrophages engulfing target cells in the hCD47-IgV^{hi} group was significantly lower than in the hCD47-IgV^{lo} group, and similarly, the hCD47-iso2^{hi} group showed a significantly lower percentage compared with the hCD47-iso2^{lo} group. These findings suggest that the resistance against phagocytosis of these cells is positively correlated with the transgenic expression levels of the mutant and wild-type CD47, and that this resistance is specifically provided by the transgenic expression of the mutant and wild-type CD47.

These results further demonstrate that the human CD47 mutant in this embodiment specifically confers protection effect to cells against macrophage phagocytosis, with its inhibitory effect on macrophage phagocytosis in vitro comparable to that of wild-type human CD47.

3. In Vitro Assessment of the Ability of Mouse CD47 Mutant, Structurally Corresponding To the Human Mutant Provided in this Embodiment, to Protect Mouse CD47KO A20 Cells from Phagocytosis by Mice Bone Marrow-Derived Macrophages, Compared to that of Wild-Type Mouse CD47

To investigate whether the phagocytosis-inhibiting ability of the CD47 mutant in this embodiment is species-specific, a mouse CD47 mutant was designed and constructed following the same strategy as the human CD47 mutant (hCD47-IgV). This involved replacing the sequences of transmembrane and intracellular domains of mouse CD47 with an attachment signal sequence of a GPI membrane anchor. Lentiviral transduction of mouse CD47KO A20 leukemia cells (generated via CRISPR/Cas9 technology) with constructs encoding the mouse CD47 mutant and its wild-type counterpart successfully yielded CD47KO A20 cells stably expressing the mouse CD47 mutant (mCD47-IgV) and the mouse CD47 wild-type control (mCD47-iso2), as shown in FIG. 3B.

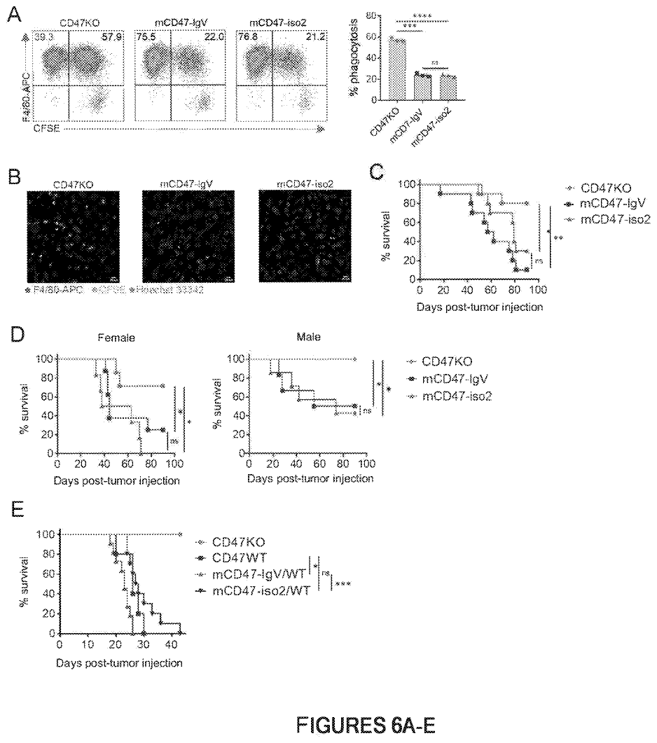

This embodiment involves obtaining BALB/c mice bone marrow cells genetically identical to A20 cells, and using them to prepare macrophages. The phagocytic activities of these macrophages towards CD47KO A20 cells and CD47KO A20 cells stably expressing mCD47-IgV/mCD47-iso2 (target cells pre-labeled with CFSE, macrophages stained with F4/80-APC antibody) were assessed using flow cytometry and confocal microscopy (the phagocytosis assays in vitro were performed as mentioned previously). FIGS. 6A-B present the results, comparing the protective effect provided by mouse CD47 mutant structurally corresponding to human mutant in vitro against macrophage phagocytosis of CD47KO A20 cells, with wild-type mouse CD47 as a reference. FIG. 6A shows the comparison of anti-phagocytic effects provided by the mouse CD47 mutant and wild-type control for CD47KO A20 cells (labeled with CFSE) in an assessment system of macrophages derived from BALB/c mice bone marrow (stained with F4/80-APC). The left panel of FIG. 6A displays representative flow cytometry profiles, and the right panel is a statistical graph of phagocytosis levels (***P<0.001; ****P<0.0001; ns indicates no significance). The x-axis of the bar graph in FIG. 6A represents groups of cells stably expressing mouse CD47 mutants (mCD47-IgV) or wild-type mouse CD47 (mCD47-iso2), while the y-axis indicates the percentages of macrophages that have phagocytosed target cells. Compared to that in the CD47KO group, the percentages of macrophages that have phagocytosed target cells is significantly reduced in the mCD47-IgV and mCD47-iso2 groups. FIG. 6B displays the results obtained using confocal imaging.

The results shown in FIGS. 6A and 6B demonstrate that mouse macrophages efficiently engulf CD47KO A20 cells in vitro. However, the phagocytosis levels of CD47KO A20 cells expressing mCD47-IgV and mCD47-iso2 by these macrophages were comparably and significantly reduced compared with that of CD47KO A20 cells. These results indicate that, consistent with findings obtained in human cell system, mouse CD47 mutant structurally corresponding to the human mutant in this embodiment can protect cells from phagocytosis by mouse macrophages to a comparable degree as the wild-type mouse CD47. Thus, both human and mouse CD47 mutants provided in this embodiment can protect CD47KO cells from macrophage phagocytosis at levels comparable to their respective wild-type counterparts.

### Embodiment 4

This embodiment describes the investigation of human CD47 mutant's ability to inhibit transplant rejection in vivo.

This embodiment employs immunodeficient NCG mice lacking functional T, B, and Natural Killer (NK) cells in experiments. Previous studies have shown that the rejection of human cell grafts in NCG mice, due to the absence of these immune cell types, is primarily mediated by macrophages. The SIRPα on NCG mouse myeloid cells exhibits functional cross-reactivity with human CD47, thereby it can be engaged by human CD47 and be activated to perform its immunosuppressive functions such as phagocytic inhibition. Therefore, this embodiment assesses the in vivo ability of the CD47 mutants of this embodiment to inhibit myeloid cell-mediated transplant rejection by comparing the tumorigenic potential of CD47KO Jurkat cells with these CD47KO Jurkat cells expressing hCD47-IgV or hCD47-iso2 in NCG mice.

The procedure involved intravenous injection of 1×10⁶ CD47KO (n=6) or hCD47-IgV-expressing (n=6) or hCD47-iso2-expressing (n=7) CD47KO Jurkat cells into NCG mice whose SIRPα is capable of cross-reacting with human CD47 to establish a leukemia model. Survival curves were compared to assess the protective abilities of the human CD47 mutant and wild-type control against phagocytosis and rejection of CD47KO Jurkat cells in vivo. Results, as depicted in FIG. 5G, show the days post-tumor cell injection on the x-axis and the survival percentages of mice on the y-axis, *P<0.05; **P<0.01; ns indicates no significance. The results in FIG. 5G reveal that all mice in the CD47KO Jurkat cell injection group exhibited long-term tumor-free survival, whereas the survival percentages in the two experimental groups receiving hCD47-IgV or hCD47-iso2-expressing CD47KO Jurkat cells intravenously were significantly lower, and the majority of recipient mice in these two groups died from tumors at similar levels. This indicates robust rejection of the CD47KO Jurkat cell grafts, which was comparably inhibited by the transgenic expression of hCD47-IgV and hCD47-iso2.

The in vivo assay results indicate that hCD47-IgV exhibits equivalent inhibitory capacity to hCD47-iso2 in suppressing rejection. Specifically, in NCG mice whose SIRPα can cross-react with human CD47, the human CD47 mutant of this embodiment effectively protects CD47KO Jurkat cell grafts from rejection at levels comparable to its wild-type counterpart.

### Embodiment 5

This embodiment describes the process of investigating the in vivo inhibitory effects of transplant rejection by mouse CD47 mutants.

The in vivo protective capacity of mouse CD47 mutant, structurally corresponding to human CD47 mutant, against phagocytosis and rejection of mouse cells was assessed in syngeneic mouse model.

Specifically, CD47KO A20 cells prepared previously, or CD47KO A20 cells stably expressing mCD47-IgV or mCD47-iso2, were injected intravenously (1×10⁶ cells per mouse) into syngeneic BALB/c mice depleted of T, B, and NK cells by sublethal irradiation (3.5 Gy, 6 hours prior to cell injection), and retaining only macrophages (FIG. 6C, n=10 per group), or into immunodeficient Rag-BALB/c mice lacking T and B cells (FIG. 6D, CD47KO, n=7 for females, n=5 for males; mCD47-IgV, n=8 for females, n=6 for males; mCD47-iso2, n=7 for females, n=7 for males). Tumor cell transplantation models were established to evaluate survival curves of different groups of mice.

FIG. 6C illustrates the survival curves of female BALB/c mice (irradiated with 3.5 Gy, 6 hours prior to cell injection) injected via the tail vein with CD47KO, mCD47-IgV, or mCD47-iso2 A20 cells to establish leukemia mouse model. The curves were compared to assess the in vivo resistance against phagocytosis and rejection provided by the mouse CD47 mutant and wild-type control for CD47KO A20 cells, *P<0.05, **P<0.01, ns indicates no significance. The x-axis represents days post-tumor cell injection, and the y-axis shows the percentages of surviving mice.

FIG. 6D presents survival curves of Rag-BALB/c mice, both female (left) and male (right), injected via the tail vein with CD47KO, mCD47-IgV, or mCD47-iso2 A20 cells to establish leukemia mouse model. These curves were compared to assess the in vivo protective effects against phagocytosis and rejection provided by the mouse CD47 mutant and wild-type control to CD47KO A20 cells, *P<0.05, ns indicates no significance. The x-axis denotes days post-tumor cell injection, and the y-axis indicates the percentages of surviving mice. Assessment of mortality data reveal that CD47KO A20 cells expressing mCD47-IgV and mCD47-iso2 exhibit comparably increased leukemogenic potential compared with CD47KO A20 cells (FIGS. 6C and 6D).

This embodiment also examined whether the transgenic overexpression of the mouse CD47 mutant, compared with that of wild-type control, could in vivo increase the resistance against phagocytosis and rejection of wild-type mouse cells possessing endogenous CD47 expression and whether the further overexpression of the mouse CD47 mutant in the context of the endogenous CD47 expression in wild-type cells provided a detectable survival advantage over the overexpression of wild-type CD47.

In aforementioned tests, mCD47-IgV and mCD47-iso2 were expressed in CD47KO A20 cells, with the transgenic expression levels of CD47 on these cells being considerably lower than those of endogenous CD47 on wild-type cells (FIG. 3B), that may cause the negative effects of CD47 transgenic expression to be indetectable. To elevate CD47 expression levels in transgenic cells above those in wild-type cells expressing only endogenous CD47, this embodiment stably transduced wild-type A20 cells to express mCD47-IgV or mCD47-iso2 (mCD47-IgV/WT, mCD47-iso2/WT). In vivo tests were conducted to verify whether the expression of the mouse CD47 mutant, compared with that of wild-type control, could further enhance the resistance of wild-type cells against phagocytosis and rejection and improve their transplant survival rate.

Specifically, four groups of cells, namely CD47KO A20 cells prepared in the previous embodiment (denoted as CD47KO, n=3), mCD47-IgV/mCD47-iso2 overexpressing wild-type A20 cells (denoted as mCD47-IgV/WT, n=11; mCD47-iso2/WT, n=10) and wild-type A20 cells (denoted as CD47WT, n=5), were separately injected via tail vein at 1×10⁶ per mouse into wild-type BALB/c mice (irradiated with 3.5 Gy, 6 hours prior to cell injection) to establish tumor cell transplantation models. Survival curves of the four groups of mice were plotted (FIG. 6E).

FIG. 6E illustrates the survival curves of the four groups of wild-type BALB/c mice (irradiated with 3.5 Gy, 6 hours prior to cell injection) injected with aforementioned A20 cells via the tail vein to construct the leukemia mouse model. These curves were compared to assess the in vivo enhancement of resistance of wild-type A20 cells against phagocytosis and rejection provided by the transgenic overexpression of the mouse CD47 mutant and wild-type control, *P<0.05, ***P<0.001, ns indicates no significance. The x-axis represents days post-tumor cell injection, and the y-axis shows the percentages of surviving mice. The mortality of mice was monitored.

Results in FIG. 6E indicate that all CD47KO A20 cell-injected mice exhibited long-term tumor-free survival, whereas all wild-type A20 cell-injected mice (CD47WT) succumbed to tumor, suggesting that endogenous CD47 significantly protects cells from phagocytosis and rejection. All mice in the mCD47-IgV/WT and mCD47-iso2/WT groups also died from tumors, with a significantly accelerated mortality in mice of mCD47-IgV/WT group compared with that in mice of CD47WT group (FIG. 6E). Despite higher level of CD47 surface expression in both CD47-IgV/WT and CD47-iso2/WT cells (co-expressing transgenic and endogenous CD47) compared with CD47WT cells expressing only endogenous CD47 (FIG. 3C), the mortality in mice of mCD47-iso2/WT group was similar to mice in CD47WT group and significantly decelerated than mice in mCD47-IgV/WT group, indicating higher tumor cell survival in mCD47-IgV/WT group. This may be due to CD47-IgV, unlike CD47-iso2, not transmitting signals of inhibitory effects on cell survival, thereby conferring a survival advantage.

The in vivo test results provided by this embodiment confirm consistency with previous findings obtained by using human cell systems and human CD47 mutant. In a mouse tumor cell transplantation model, mouse CD47 mutant protect CD47KO A20 cells from phagocytosis and rejection as effectively as its wild-type CD47 counterpart. This indicates that CD47 mutants of different species (e.g., human/mouse) with the structural characteristics of the mutant provided by the present disclosure exhibit comparable phagocytosis inhibition function compared to their respective wild-type counterparts. Additionally, these in vivo test results also demonstrate that, when mCD47-IgV and mCD47-iso2 are transgenically overexpressed in wild-type cells possessing endogenous CD47 expression, mCD47-IgV further enhances the resistance of wild-type cells against phagocytosis and rejection, thereby increasing tumor cell survival rates. In contrast, mCD47-iso2 does not improve tumor cell survival rates. This confirms in vivo that the transgenic expression of CD47 mutants can provide higher graft survival rates compared with that of wild-type CD47.

### Embodiment 6

This embodiment investigates whether overexpression of the aforementioned mutant affects cell function by assessing the engraftment and differentiation potential of mouse hematopoietic stem/progenitor cells (HSPCs) stably expressing mCD47-IgV and its wild-type control (mCD47-iso2) in syngeneic recipient mice.

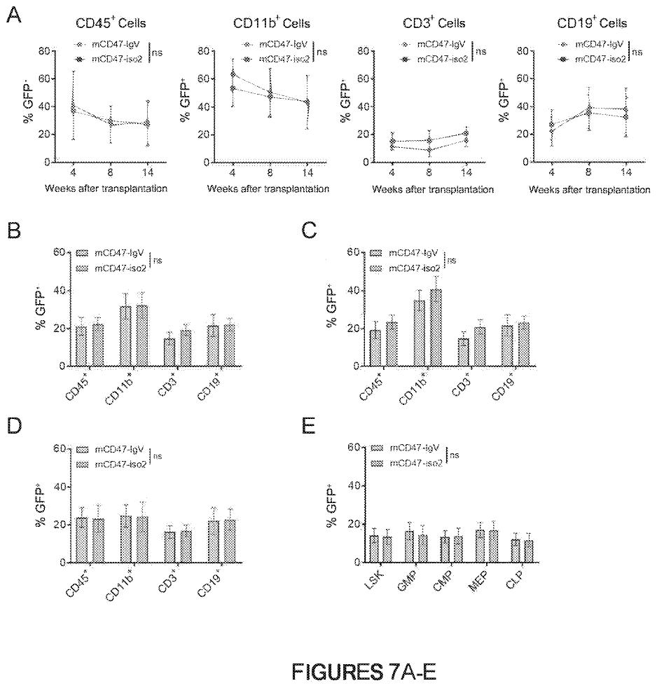

The procedure involves harvesting bone marrow cells from 8-10-week-old GFP transgenic C57BL/6 mice (from femur, tibia, and ilium) with endogenous CD47 knockout (CD47KO). These cells were purified by removing lineage marker-positive (Lin⁺) cells using magnetic beads coupled with a cocktail of antibodies, followed by flow cytometry sorting to isolate Sca-1⁺c-Kit⁺lineage⁻ (LSK) hematopoietic stem/progenitor cells, which include hematopoietic stem cells (HSCs) and multipotent progenitors (MPPs). The obtained LSK cells were transduced with lentiviruses encoding mouse CD47-IgV mutant or wild-type CD47 (MOI=10) in LSK culture medium containing 5 μg/ml polybrene and subsequently cultured in vitro for 60 hours. GFP-negative, 8-10-week-old syngeneic wild-type C57BL/6 male recipient mice were preconditioned with a lethal dose of 9 Gy total body irradiation and transplanted 6 hours later with 5×10⁴ GFP⁺ mCD47-IgV- or mCD47-iso2-transduced CD47KO LSK cells and 3×10⁵ carrier cells per mouse (i.e., bone marrow cells of GFP-negative recipient mice) via tail vein injection. Peripheral blood cells were collected at indicated time points (4, 8, and 14 weeks post-transplantation) for GFP⁺ donor cell chimerism analysis by flow cytometry. At 14 weeks, the recipient mice were sacrificed, and the chimerism levels and lineage distribution of GFP⁺ cells derived from donor hematopoietic stem/progenitor cells in spleen, lymph nodes, and bone marrow were analyzed using flow cytometry. Results are shown in FIG. 7.

The LSK culture medium composition is as follows: IMDM complete medium supplemented with Recombinant Mouse SCF Protein (mSCF) at 100 ng/ml, Recombinant Mouse Flt-3 ligand Protein (mFlt3-L) at 50 ng/ml, and Recombinant Mouse Thrombopoietin Protein (mTPO) at 10 ng/ml.

FIG. 7A illustrates the chimerism and lineage distribution of GFP-positive donor-derived cells in the peripheral blood of the two groups of recipient mice receiving transgenic hematopoietic stem/progenitor cells (mCD47-IgV, mCD47-iso2, n=8 per group) analyzed at indicated time points (4, 8, and 14 weeks post-transplantation). The x-axis represents weeks after transplantation, and the y-axis indicates the percentage of GFP-positive cells. Results in FIG. 7A indicate no detectable differences in the levels of GFP-positive, donor-derived CD45⁺ cells, CD11b⁺ myeloid cells, CD3⁺ T cells, or CD19⁺ B cells between the two recipient groups transplanted with mCD47-IgV-transduced or mCD47-iso2-transduced LSK cells.

At week 14 post-transplant, the aforementioned recipient mice were sacrificed, and donor chimerism was assessed in the spleen, lymph nodes, and bone marrow. FIG. 7B shows the chimerism percentages of different cell lineages derived from donor LSK cells in the recipient spleen between the two experimental groups at 14 weeks post-transplant; FIG. 7C illustrates the chimerism percentages in the recipient lymph nodes; FIG. 7D depicts the chimerism percentages in the recipient bone marrow. The x-axis in FIGS. 7B-D represents cell types, and the y-axis indicates the percentage of GFP-positive cells. Results indicate no difference in donor chimerism levels and lineage distributions between the two recipient groups transplanted with wild-type or mutant CD47-transduced LSK cells. Additionally, FIG. 7E presents the percentages of GFP⁺ donor-derived LSK, GMP, CMP, MEP, and CLP cells in bone marrow of the recipient mice, showing similar levels of these cells in both experimental groups.

In summary, the results of this embodiment demonstrate that the mutant CD47 expression, compared with wild-type CD47 expression, does not affect HSPC function. Additionally, the long-term survival of CD47KO cells transgenically expressing the mouse CD47 mutant (mCD47-IgV) in vivo further supports the protective effect of mCD47-IgV against phagocytosis.

### Embodiment 7

The embodiment evaluates the protective effect of transgenic expression of the human CD47 mutant on porcine epithelial cells PK15 against human macrophage phagocytosis in vitro.

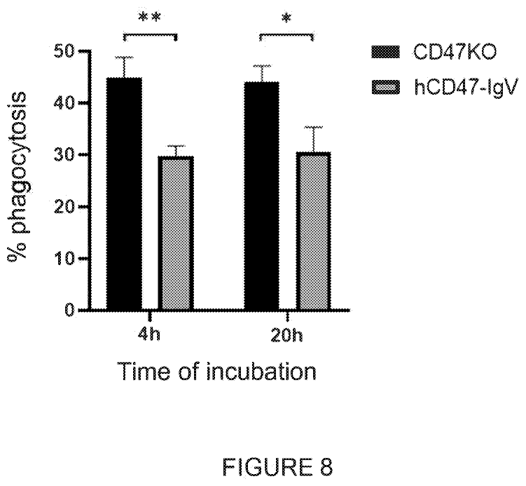

The procedure involves isolating human PBMCs using the aforementioned method, culturing them in RPMI 1640 complete medium supplemented with 20 ng/ml human M-CSF for 7 days to induce macrophage differentiation, with medium replacement every 2-3 days during this process. Harvested macrophages (2×10⁵ cells/well) were seeded in 12-well plates, cultured for 16 hours, then switched into serum-free medium for an additional 2 hours. Target cells, namely CD47KO PK15 epithelial cells (denoted as CD47KO in the figure) and hCD47-IgV expressing CD47KO PK15 epithelial cells (denoted as hCD47-IgV in the figure), were labeled with 2×10⁻⁶ M PKH67 dye according to the manufacturer's protocol. These labeled target cells were then added to the wells containing the aforementioned macrophages (effector cells) at a 1:2 effector-to-target ratio. After 4 or 20 hours, cells were collected by trypsinization, and macrophages were stained with anti-human CD172α/β (SIRPα/β)-APC antibody. Phagocytosis was quantified by flow cytometry, measuring the percentages of SIRPα⁺PKH67⁺ macrophages that have phagocytosed target cells. The results are shown in FIG. 8, which analyzes the phagocytosis of the two groups of target cells by macrophages.

The x-axis of FIG. 8 represents the incubation time of macrophages with target cells, and the y-axis shows the percentages of macrophages that have engulfed target cells. The results indicate that after incubation for 4 hours, the percentage of macrophage engulfment of target cells in the hCD47-IgV group was significantly lower than that in the CD47KO group. Similarly, after incubation for 20 hours, the engulfment percentage in the hCD47-IgV group was also significantly lower compared with the CD47KO group. Specifically, CD47KO PK15 cells expressing hCD47-IgV are significantly more resistant to phagocytosis compared with CD47KO PK15 cells. These findings demonstrate that the transgenic expression of the aforementioned human CD47 mutant can protect porcine kidney epithelial cells PK15 from phagocytosis by human macrophages, indicating that the human CD47 mutant can shield porcine cells from human macrophage engulfment.

### Embodiment 8

This embodiment provides an assessment of the angiogenic potential of CD47KO EA.hy926 cells stably expressing human CD47 mutant or wild-type control, as well as CD47KO and WT EA.hy926 cells, both in vitro and in vivo.

Given that wild-type CD47 is known to inhibit angiogenesis and blood flow, thereby impairing graft survival and function, this embodiment evaluates the impact of CD47 mutant on angiogenesis using a tube formation assay. The assay compares the in vitro angiogenic potential of four cell groups: namely CD47KO EA.hy926 endothelial cells stably expressing the human CD47 mutant (hCD47-IgV), those stably expressing human wild-type CD47 (hCD47-iso2), wild-type EA.hy926 (CD47WT), and CD47KO EA.hy926 (CD47KO) endothelial cells.

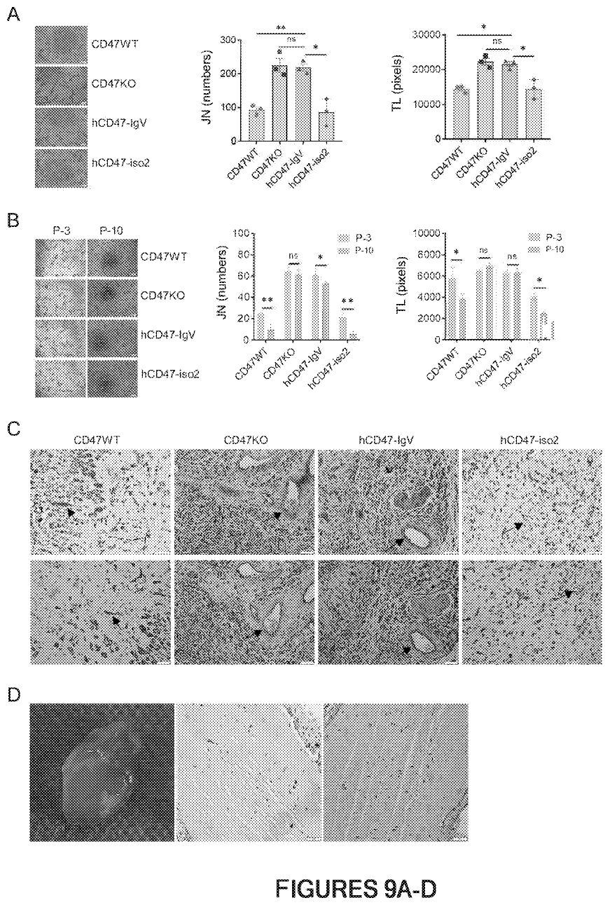

The procedure involves adding 50 μl of Matrigel Matrix to each well of a 96-well plate, which was then allowed to polymerize into gel at 37 °C for 30 minutes. Subsequently, endothelial cells (ECs) were plated at 5.5×10⁴ cells per well onto the surface of the gelled matrix. The cells were cultured at 37 °C and 5% C02 for 6 hours. Images of the tube formation were acquired, then three fields per experimental group were randomly selected and analyzed using the Angiogenesis Analyzer plugin of ImageJ software to obtain the parameters of junction number (JN) and total length (TL), which were used to assess angiogenesis, as shown in FIG. 9.

FIG. 9A presents the results of an in vitro tube formation assay for the four groups of cells. The left panel shows representative images of tube formation, while the right panel displays statistical graphs of junction numbers and total length of tube-like structures within three randomly selected fields of each group analyzed using ImageJ software (plugin: Angiogenesis Analyzer), *P<0.05, **P<0.01, ns indicates no significance. Consistent with previous reports on the inhibitory role of wild-type CD47 in angiogenesis (Gao, Q et al. 2016. Cell Death Dis. 7, e2368; Meijles, D N et al. 2017. Sci Signal 10, eaaj1784), results in FIG. 9A demonstrates that compared with wild-type EC group (denoted as CD47WT in the figure), CD47KO and hCD47-IgV groups exhibit comparably and significantly increased junction numbers, up to twice or more than those of CD47WT group. Additionally, the total length of tube-like structures in CD47KO and hCD47-IgV groups is comparably and significantly greater than in CD47WT group. In contrast, the hCD47-iso2 group shows junction number and total length of tube-like structures comparable with CD47WT group and significantly lower than hCD47-IgV group. These findings indicate that CD47KO ECs have significantly improved angiogenesis compared with CD47WT ECs, and hCD47-IgV-overexpressing ECs also show significantly stronger angiogenic capacity than CD47WT ECs, comparable to CD47KO ECs. Furthermore, the angiogenic capacity of hCD47-iso2-overexpressing ECs is significantly lower than that of hCD47-IgV-overexpressing ECs, with no detectable difference in comparison to that of CD47WT ECs.

FIG. 9B presents the angiogenic capacity of the four aforementioned groups of ECs at passages 3 (P-3) and passages 10 (P-10) undergoing continuous passage after thawing. The left panel displays representative tube formation images, while the right panel shows statistical graphs of EC junction numbers and total length within three random fields for each group, *P≤0.05, **P<0.01, and ns indicates no significance. The results indicate a significant reduction in angiogenic capacity due to multiple passages and senescence in both wild-type and hCD47-iso2-overexpressing ECs in vitro, evidenced by a significant decrease in junction number (JN) and total length (TL) for P-10 cells compared with P-3 cells. In contrast, no such reduction was observed in the CD47KO EC group, and the hCD47-IgV EC group of this embodiment showed minimally detected reduction.

This embodiment also evaluated the in vivo angiogenic capacity of the four aforementioned groups of endothelial cells using the Matrigel plug assay.

The procedure involves mixing 5×10⁶ ECs of each cell group with 400 μl Matrigel Matrix and injecting these mixtures subcutaneously into the inguinal region of 8-10-week-old NCG mice. After 10 days, the Matrigel plugs were retrieved and analyzed for in vivo neovascularization using hematoxylin and eosin (H&E) staining and immunohistochemical (IHC) staining with anti-human CD31 antibody. FIG. 9C displays the H&E staining (upper panel) and anti-human CD31 IHC staining results (lower panel) of newly formed vessels in Matrigel plugs separately containing different groups of ECs and subcutaneously implanted in NCG mice, with arrows indicating typical vessels. FIG. 9D shows the autopsy photograph (left panel), H&E staining (middle panel), and anti-human CD31 IHC staining results (right panel) of Matrigel plugs without ECs subcutaneously implanted in NCG mice and used for the blank control. The results in FIGS. 9C and 9D indicate that no microvessel formation was detected in the Matrigel plugs without ECs, suggesting that the newly formed vessels in the four experimental groups were specifically developed by the human ECs mixed in the gel plugs, not by the migrated recipient mouse ECs. Specifically, compared with Matrigel plugs with wild-type ECs, plugs with CD47KO ECs showed significantly increased CD31⁺ ECs and microvessel formation. Similarly, Matrigel plugs with hCD47-IgV-overexpressing ECs also exhibited a significant increase in angiogenesis to a comparable extent as plugs with CD47KO ECs, while there was no difference in levels of CD31⁺ ECs or microvessel formation between plugs with hCD47-iso2-overexpressing ECs and plugs with wild-type ECs (FIG. 9C).

In summary, both the in vitro and in vivo data provided by this embodiment demonstrate that, different from the endogenous wild-type CD47 expression and transgenic overexpression of hCD47-iso2, transgenic overexpression of the hCD47-IgV mutant on ECs does not inhibit EC angiogenesis and ameliorates cellular senescence induced by continuous passage to a similar extent as that of CD47KO ECs. Thus, the hCD47-IgV mutant provided by this embodiment does not suppress neovascularization.

It is to be noted that the particular embodiments described previously are exemplary. People skilled in the art, with inspiration from the present disclosure, would be able to devise various solutions, all of which fall within the scope of the present disclosure and are protected by it. Further, people skilled in the art would appreciate that the description and accompanying drawings provided herein are illustrative and form no limitation to any of the appended claims. The scope of the present disclosure is defined by the appended claims and equivalents thereof. The present disclosure provided herein encompasses multiple inventive concepts, indicated by phrases such as “preferably” and “according to a preferred embodiment”, each denoting a distinct concept disclosed in the respective paragraph. The applicant reserves the right to file one or more divisional applications based on each inventive concept. Throughout the present disclosure, features introduced by the terms “preferably” and “according to a preferred embodiment” are optional but not mandatory, and the applicant also reserves the right to withdraw or delete such preferred features at any time.

1-15. (canceled)

16. A CD47 mutant, comprising a partial amino acid sequence of CD47 and an amino acid sequence of a glycosylphosphatidylinositol (GPI) attachment signal.

17. The CD47 mutant of claim 16, wherein the CD47 mutant eliminates negative effects promoting graft injury by removing amino acid sequences of CD47 transmembrane region and intracellular region, while adding the amino acid sequence of GPI attachment signal.

18. The CD47 mutant of claim 16, wherein the CD47 mutant is obtained by fusing the partial amino acid sequence of CD47 with the amino acid sequence of the GPI attachment signal.

19. The CD47 mutant of claim 16, wherein the partial amino acid sequence of CD47 comprises its signal peptide and extracellular IgV domain sequence, preferably the partial amino acid sequence of CD47 comprises residues 1-137 of human CD47 or residues 1-161 of mouse CD47.

20. The CD47 mutant of claim 16, wherein the amino acid sequence of the GPI attachment signal is fused to the carboxyl terminus of the CD47 protein, thereby enabling the mutant to possess a GPI membrane anchor, preferably the amino acid sequence of the GPI attachment signal is composed of 37 amino acids from the carboxyl terminus of the decay-accelerating factor.

21. The CD47 mutant of claim 20, wherein the GPI membrane anchor is a GPI membrane anchor specifically enriched in lipid rafts.

22. The CD47 mutant of claim 16, wherein the CD47 mutant achieves proper intracellular trafficking and high expression on the outer leaflet of the cell membrane via the fused GPI membrane anchor.

23. The CD47 mutant of claim 16, wherein the CD47 mutant, when overexpressed in donor cells, can inhibit transplant rejection mediated by recipient myeloid cells and other SIRPα-positive immune cells.

24. The CD47 mutant of claim 16, wherein the CD47 mutant is a human CD47 mutant comprising an amino acid sequence of SEQ ID NO.1, preferably the amino acid sequence of the human CD47 mutant is obtained by fusing residues 1-137 of human CD47 with the GPI attachment signal sequence consisting of 37 amino acids from the carboxyl terminus of the decay-accelerating factor.

25. The CD47 mutant of claim 16, wherein the CD47 mutant is a mouse CD47 mutant comprising an amino acid sequence of SEQ ID NO.2, preferably the mouse CD47 mutant is obtained by fusing residues 1-161 of mouse CD47 with the GPI attachment signal sequence consisting of 37 amino acids from the carboxyl terminus of the decay-accelerating factor.

26. A humanized animal transgenically expressing the CD47 mutant of claim 16.

27. The humanized animal of claim 26, wherein the humanized animal is a pig and/or a non-human primate.

28. The humanized animal of claim 26, wherein the non-human primate is a monkey, ape, or baboon.

29. A method for treating a disease or condition in a subject, comprising introducing hypoimmunogenic transgenic cell grafts, tissue grafts, organ grafts or hypoimmunogenic allogeneic cell therapy products expressing the CD47 mutant of claim 16 into the subject.

30. The method of claim 29, wherein the hypoimmunogenic transgenic cell grafts or hypoimmunogenic allogeneic cell therapy products include hematopoietic stem cells, islet cells, embryonic stem cells, induced pluripotent stem cells, or stem cell-differentiated derivatives.

31. A xenotransplantation method, comprising introducing cells, tissues, or organs obtained from transgenic animals expressing the CD47 mutant of claim 16 into a subject, optionally into humans.

32. A xenotransplantation method, comprising introducing cells, tissues, or organs obtained from the humanized animal of claim 26 into a subject, optionally into humans.

33. A method for production and preparation of hypoimmunogenic transgenic cell grafts, tissue grafts, organ grafts or hypoimmunogenic allogeneic cell therapy products, comprising introducing the CD47 mutant of claim 16 into the hypoimmunogenic transgenic cell grafts, tissue grafts, organ grafts or hypoimmunogenic allogeneic cell therapy products.

34. The method of claim 33, wherein the hypoimmunogenic transgenic cell grafts, tissue grafts, organ grafts or hypoimmunogenic allogeneic cell therapy products include hematopoietic stem cells, islet cells, embryonic stem cells, induced pluripotent stem cells, or stem cell-differentiated derivatives.

35. A method for production and preparation of hypoimmunogenic transgenic cell grafts, tissue grafts, organ grafts or hypoimmunogenic allogeneic cell therapy products, comprising obtaining the hypoimmunogenic transgenic cell grafts, tissue grafts, organ grafts or hypoimmunogenic allogeneic cell therapy products from the humanized animal of claim 26.


SEQUENCE LISTING (30 sequences)

SEQ ID NO: 1 - protein, 174 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFPNKGSGTTSGTTRLLSGHTCFTLTGLLGTLVTMGLLT

SEQ ID NO: 2 - protein, 198 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKPNKGSGTTSGTTRLLSGHTCFTLTGLLGTLVTMGLLT

SEQ ID NO: 3 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 4 - protein, 324 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 5 - protein, 137 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWF

SEQ ID NO: 6 - protein, 161 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEK

SEQ ID NO: 7 - protein, 37 aa, Homo sapiens
PNKGSGTTSGTTRLLSGHTCFTLTGLLGTLVTMGLLT

SEQ ID NO: 8 - DNA, 525 bp, Homo sapiens
  source 1..525: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggtttccaaataaaggaagtggaaccacttcaggtactacccgtcttctatctgggcacacgtgtttcacgttgacaggtttgcttgggacgctagtaaccatgggcttgctgacttag

SEQ ID NO: 9 - DNA, 597 bp, Mus musculus
  source 1..597: mol_type = other DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagccaaataaaggaagtggaaccacttcaggtactacccgtcttctatctgggcacacgtgtttcacgttgacaggtttgcttgggacgctagtaaccatgggcttgctgacttag

SEQ ID NO: 10 - DNA, 114 bp, Homo sapiens
  source 1..114: mol_type = other DNA
ccaaataaaggaagtggaaccacttcaggtactacccgtcttctatctgggcacacgtgtttcacgttgacaggtttgcttgggacgctagtaaccatgggcttgctgacttag

SEQ ID NO: 11 - DNA, 411 bp, Homo sapiens
  source 1..411: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttt

SEQ ID NO: 12 - DNA, 483 bp, Mus musculus
  source 1..483: mol_type = other DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaag

SEQ ID NO: 13 - DNA, 918 bp, Homo sapiens
  source 1..918: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 14 - DNA, 975 bp, Mus musculus
  source 1..975: mol_type = other DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtga

SEQ ID NO: 15 - protein, 18 aa, Homo sapiens
MWPLVAALLLGSACCGSA

SEQ ID NO: 16 - DNA, 54 bp, Homo sapiens
  source 1..54: mol_type = other DNA
atgtggcccctggtagcggcgctgttgctgggctcggcgtgctgcggatcagct

SEQ ID NO: 17 - protein, 119 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWF

SEQ ID NO: 18 - DNA, 357 bp, Homo sapiens
  source 1..357: mol_type = other DNA
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttt

SEQ ID NO: 19 - protein, 18 aa, Mus musculus
MWPLAAALLLGSCCCGSA

SEQ ID NO: 20 - DNA, 54 bp, Mus musculus
  source 1..54: mol_type = other DNA
atgtggcccttggcggcggcgctgttgctgggctcctgctgctgcggttcagct

SEQ ID NO: 21 - protein, 143 aa, Mus musculus
QLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEK

SEQ ID NO: 22 - DNA, 429 bp, Mus musculus
  source 1..429: mol_type = other DNA
caactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaag

SEQ ID NO: 23 - DNA, 1221 bp, Homo sapiens
  source 1..1221: mol_type = other DNA
aagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccgccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggtttccaaataaaggaagtggaaccacttcaggtactacccgtcttctatctgggcacacgtgtttcacgttgacaggtttgcttgggacgctagtaaccatgggcttgctgacttaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcacgcaacgtggcgtggtgtgcactgtgtttgctgacgcaacccccactggttggggcattgccaccacctgtcagctcctttccgggactttcgctttccccctccctattgccacggcggaactcatcgccgcctgccttgc

SEQ ID NO: 24 - DNA, 1090 bp, Homo sapiens
  source 1..1090: mol_type = other DNA
taaccaatcagttcgcttctcgcttctgttcgcgcgcttctgctccccgagctcaataaaagagcccacaacccctcactcggcgcgccgccaccatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgagtcgacaatcaacctctggattacaaaatttgtgaaagattgactggtattcttaactatgttgctcctttacgcta

SEQ ID NO: 25 - DNA, 1084 bp, Mus musculus
  source 1..1084: mol_type = other DNA
aggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacgtgaaatgaccctgtgccttatttgactaaccaatcagttcgcttctcgcttctgttcgcgcgcttctgctccccgagctcaataaaagagcccacaacccctcactcggcgcgcctgccaccatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagccaaataaaggaagtggaaccacttcaggtactacccgtcttctatctgggcacacgtgtttcacgttgacaggtttgcttgggacgctagtaaccatgggcttgctgacttaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtg

SEQ ID NO: 26 - DNA, 1157 bp, Mus musculus
  source 1..1157: mol_type = other DNA
ttatttgactaaccaatcagttcgcttctcgcttctgttcgcgcgcttctgctccccgagctcaataaaagagcccacaacccctcactcggcgcgcctgccaccatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgagtcgacaatcaacctctggattacaaaatttgtgaaagattgactggtattcttaactatgttgctcctatacgcta

SEQ ID NO: 27 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 28 - RNA, 20 nt, synthetic construct
  source 1..20: mol_type = other RNA
ctactgaagtatacgtaaag

SEQ ID NO: 29 - RNA, 20 nt, synthetic construct
  source 1..20: mol_type = other RNA
ttggcggcggcgctgttgct

SEQ ID NO: 30 - RNA, 20 nt, synthetic construct
  source 1..20: mol_type = other RNA
aacggtgaattctacagatt